(19) United States
(12) Patent Application Publication
(10) Pub. No.: US 2025/0000738 A1
(43) Pub. Date: Jan. 2, 2025

(54) SYSTEMS AND METHODS FOR TRACKING REAL-TIME FORCE-MOTION PATTERNS OF MASSAGE STROKE TYPES USING HANDHELD DEVICE-ASSISTED QUANTIFIABLE SOFT TISSUE MANIPULATION

(71) Applicant: The Trustees of Indiana University, Bloomington, IN (US)

(72) Inventors: Abhinaba Bhattacharjee, Indianapolis, IN (US); Stanley Y.P. Chien, Zionsville, IN (US); Sohel Anwar, Zionsville, IN (US); M. Terry Loghmani, Indianapolis, IN (US)

(21) Appl. No.: 18/705,090

(22) PCT Filed: Oct. 31, 2022
(86) PCT No.: PCT/US2022/078958

§ 371 Date: Apr. 26, 2024

Related U.S. Application Data
(60) Provisional application No. 63/273,772, filed on Oct. 29, 2021.
(60) Provisional application No. 63/381,449, filed on Oct. 28, 2022.

(51) Int. Cl. A61H 7/00; G16H 15/00; G16H 20/30; G16H 40/67

(52) U.S. Cl. CPC A61H 7/003; G16H 15/00; G16H 20/30; G16H 40/67; A61H 2201/5007; A61H 2201/5043; A61H 2201/5061; A61H 2201/5069

(57) ABSTRACT

Disclosed is a Quantifiable Soft Tissue Manipulation (QSTM) system that includes one or more force-motion applicator(s) or QSTM device(s) for digitally characterizing soft-tissue manipulation (STM) by classifying sensor data from one or more applied soft-tissue manipulation stroke types as identical and/or distinctly specific signatures of force-motion waveform patterns. The processing unit of QSTM device(s) compute quantifiable metrics which are transmitted to a remote/edge display device running a software (Q-Ware) for interactive visual graphical display, treatment recording, documentation and further analyses. The treatment metrics are further distinguished into treatment bursts and stroke counts along with classifying STM stroke type(s) as signature multi-modal force-motion waveform patterns. Eventually the classified signature forcemotion waveform patterns of associated STM stroke types from the treatment database are used to compare the degree of variabilities in identical stroke types performed by similar or different users in the form of percentage match of superimposed identical waveforms.

### CROSS-REFERENCE TO RELATED APPLICATIONS

The present application claims priority to U.S. Provisional Application No. 63/273,772, filed Oct. 29, 2021, and U.S. Provisional Application No. 63/381,449, filed Oct. 28, 2022, the complete disclosures of which are expressly incorporated by reference herein in their entireties.

### GOVERNMENT SUPPORT

This invention was made with government support under AT011494 awarded by National Institutes of Health. The Government has certain rights in the invention.

### FIELD OF THE DISCLOSURE

The present disclosure relates to tracking and characterizing a quantified force-motion pattern applied to soft tissue during real-time handheld mechatronic device-assisted therapeutic massage or soft tissue manipulation.

### BACKGROUND

Massage-based therapies, such as soft tissue mobilization or manipulation (“STM”), may be used for improving soft tissue quality in patients with acute injuries, chronic injuries, and/or diseases (e.g., knee pain, plantar fasciitis, carpal tunnel syndrome). For example, massage-base therapies may improve the structure, function, and/or the blood flow of the cells at a specific portion of soft tissue.

One such massage-based therapy is instrument-assisted soft tissue manipulation (“IASTM”), in which a physical therapist, occupational therapist, chiropractor, doctor, athletic trainer, and/or any other professional trained in massage applies pressure to the soft tissue (e.g., muscle, tendon, ligament, and/or fascia) of a patient with a rigid device. Cells within the soft tissue are load sensitive and massage-based therapies, such as IASTM, are forms of mechanotherapy which provide direct mechanical stimuli to the cells to promote endogenous tissue healing, repair, and regeneration.

However, IASTM therapies are not uniformly applied to specific injuries or parts of the patient's body because the pressure applied to the soft tissue is dependent upon the person applying the pressure. This makes IASTM and other massage-based therapies difficult to replicate, compare, determine the treatment effect, or monitor progress such that the patient may not receive consistent, progressive, or optimized care for a particular injury or disease. “Patient” may refer to both humans and animals who may be under clinical care and/or research subjects enrolled in a research protocol. It is useful to minimize differences in the application of STM by different therapists, doctors, clinicians, or practitioners and also is useful to minimize differences in the application of STM by the same therapist, doctor, or clinician between different, therapy sessions. As such, there is a need for a system and/or method for quantifying the force and motion applied to soft tissue through massage-based therapies.

### SUMMARY

The present disclosure describes methods, devices, and systems for quantifying the 3D force and motion applied to soft tissue through massage-based therapies using handheld mechatronic tools/devices with sensory nodes, for example at least a force magnitude and an angular motion in one or more dimensions with respect to a time duration, accompanying targeted mechanical stimulation, mobilization or manipulation on a soft tissue surface at a rate/frequency repeating along its motion trajectory through manually operated (hand-held) mechatronic device assisted soft tissue manipulation in real time. The methods, devices, and systems of the present disclosure facilitates tracking and characterizing a quantified force-motion pattern of an identifiable soft tissue manipulation stroke type applied to a soft tissue during real-time handheld mechatronic device assisted quantifiable therapeutic massage or soft tissue manipulation during a clinical manual therapy treatment. The system configured for such quantification and characterization of manual therapy may be referred to as a quantifiable soft tissue manipulation (“QSTM”, trademarked under U.S. Reg. No. 6839703, registered Sep. 6, 2022, registrant: Health Smart Technologies, Inc.) system. The disclosed innovations of this disclosure include handheld mechatronic processing devices (manually operated QSTM force-motion applicator units) and computer-implemented processes (e.g., firmware, software, algorithms) which perform the following:
a) graphical analysis of quantified temporal multimodal force-motion waveforms of manually operated mechatronic QSTM devices address STM motions performed in a particular session which may be used to identify and characterize STM Stroke types for replication of soft tissue manual therapy treatment, including:
i. classifying temporal multimodal force-motion waveforms of associated STM stroke types, into identical or distinctly specific signatures of force-motion waveform patterns comprising the STM treatment.
ii. accurately identifying and counting treatment strokes and bursts to avoid false positive and negative counts during random indeterministic STM motion patterns inherent to individualized patient care,
iii. assisting automatic adaptive device calibration for force and motion sensing by gravity correction for noise minimization; and
b) dynamic comparison of present and past temporal multimodal force motion waveforms from history of recorded therapeutic STM treatments to analyze degree of agreement in similar STM stroke types, in the form of percentage match in identical force-motion waveform signature patterns, of inter and intra therapist(s) performances to assess treatment consistency of adequate therapeutic practice and enhance fidelity of manual therapy.

Advantageously, the present disclosure facilitates comparing the force and motion applied to soft tissue through massage-based therapies as quantified, in order to expand the fidelity of practice. Technological development of instrument-assisted soft tissue manipulation (IASTM) tools into manually operated sensor-based handheld massage devices to quantify force and motion of STM beneficially advances the current state-of-the-art therapeutic practice in the form of QSTM. Such tools also beneficially offer real-time numeric or graphical visual feedback to the clinician in order to assist in guiding targeted dosing of mechanical loads and motion direction during massage-based therapies to curb practice subjectivity and assess functional outcomes of soft tissue manual therapy. The dynamically quantified force and motion of the handheld mechatronic device assisted soft tissue manipulation may be digitally represented in the form of temporal waveforms of force and/or angular orientation. These waveforms can be further characterized into particular force-motion waveform patterns to represent the physically applied massage stroke types on the soft tissue, which in turn may address the reproducibility or fidelity of soft tissue massage or manipulation and assist is minimizing the subjectivity and/or variabilities present in current state of art manual therapy.

QSTM systems as disclosed herein include at least one QSTM device for quantifying soft tissue manipulation. The QSTM device is configured to apply a massage therapy on a soft tissue of a patient, wherein the massage therapy includes one or more soft-tissue manipulation stroke types applied by a single practitioner during a treatment session, wherein the QSTM device is a handheld mechatronic force-motion applicator. The QSTM device may include: at least one treatment edge; at least one force or motion sensing unit mechanically coupled to the at least one treatment edge and configured to measure quantifiable metrics including 3D motions and magnitude of compressive and shear forces applied during directional hand movements of the QSTM device for the soft-tissue manipulation by performing the one or more soft-tissue manipulation stroke types using the at least one treatment edge; a processing unit coupled with the at least one force or motion sensing unit to compute resultant (RMS) force data from the measured compressive and shear forces, and angular orientation data from the 3D motions including one or more of: linear acceleration, angular velocity, or compass direction; a memory unit configured to store sensor calibration information and to record data of the compressive and shear forces as measured by the at least one force or motion sensing unit, and treatment timestamps, in order to quantify the one or more soft-tissue manipulation stroke types performed on the patient; a control button to be operated by the practitioner to change operation modes or working states of the QSTM device during the treatment session; RGB-LED lights to visualize the operation modes and the working states of the QSTM device; and a transmitter-receiver for serial communication and transmission of the RMS force and angular orientation data, the treatment timestamps, and the data of the compressive and shear forces via data stream to a remote device.

In some examples, the QSTM system may further include the remote device with an interactive visual display having multiprocessing computational and memory capacities. The remote device may be configured to: receive the data of the compressive and shear forces, the RMS force and angular orientation data, and the treatment timestamps transmitted from the at least one QSTM device; generate, based on the RMS force and angular orientation data, graphical data in form of multimodal graphical waveforms for a visual, numeric, or statistical comparison of the quantifiable metrics associated with the one or more soft-tissue manipulation stroke types performed by the practitioner during the treatment session; and execute a software program for QSTM-based electronic treatment record to generate treatment reports and to document the treatment sessions.

In some examples, the remote device may save and record the generated treatment reports and the quantifiable metrics of the massage therapy in one or more treatment sessions performed by one or more practitioner on corresponding patients for treatment data organization and maintenance. In some examples, the remote device may determine one or more burst counts of a progression of soft-tissue manipulation strokes from the multimodal graphical waveforms and corresponding stroke counts applied in different stroke frequencies as a part of the treatment reports of the treatment session.

In some examples, each of the burst counts may be determined based on one or more of: (a) computed 3D force measurements including compressive, shear lateral, and shear longitudinal force measurements, (b) angular motion measurements including yaw, pitch, and roll measurements, (c) data of the 3D motions including 3D accelerometer values, 3D gyroscope values, or 3D magnetometer values, or (d) start and stop timestamps of the treatment session. In some examples, each of the stroke counts may be determined based on thresholds of a decision tree comprising one or more of: computed 3D force measurements including compressive, shear lateral, and shear longitudinal force measurements, or start and stop timestamps associated with the burst counts.

In some examples, the generated graphical data may include a first multimodal graphical waveform representing a first soft-tissue manipulation stroke type and a second multimodal graphical waveform representing a second soft-tissue manipulation stroke type, and the visual comparison is displayed such that the first multimodal graphical waveform is superimposed on the second multimodal graphical waveform to identify a degree of variability or similarity between the multimodal graphical waveforms representing individual or identical soft-tissue manipulation stroke types performed by a same practitioner or different practitioners.

In some examples, the remote device may: classify individual soft-tissue manipulation stroke types as force-motion signature patterns of the multimodal graphical waveforms, based on the graphical features generated in the multimodal graphical waveforms associated with the plurality of soft-tissue manipulation stroke types; identify that the classified force-motion signature patterns of a first tissue manipulation stroke type and a second soft-tissue manipulation stroke type are identical or different; and generate a percentage of match of the multimodal graphical waveforms of the first and second soft-tissue manipulation stroke types in response to identifying that the first and second soft-tissue manipulation stroke types are identical based on the degree of variability or similarity estimated in the graphical features of the multimodal graphical waveforms.

In some examples, the QSTM system may further include: a first QSTM device for recording and quantifying a first treatment sub-session by facilitating the practitioner to apply the one or more soft-tissue manipulation stroke types using the first QSTM device, and a second QSTM device for recording and quantifying a second treatment sub-session by facilitating the practitioner to apply the one or more soft-tissue manipulation stroke types using the second QSTM device. The first and second QSTM devices are used one after another in sequential repetition by the same practitioner to perform a multiple-device treatment session covering regional areas of the body, and the practitioner is prompted to document treatment remarks about the treatment sub-sessions on the remote device via an interactive visual display before saving the treatment report of the performed treatment session. In some examples, the remote device is configured to display a visual feedback, automatically detect which one of the first and second QSTM devices is in use, and switch, based on the detecting without any user input, a device-specific user interface to display a live animated graphical visualization. In some examples, at least one QSTM device facilitates the practitioner to apply the one or more soft-tissue manipulation stroke types by the same QSTM device performing a single-device treatment session.

In some examples, the QSTM system further comprises a first QSTM device and a second QSTM device. The system facilitates a multiple-device treatment session by the practitioner in which a first soft-tissue manipulation is performed using the first QSTM device and a second soft-tissue manipulation is performed using the second QSTM device by the same practitioner after completion of the first soft-tissue manipulation. Each of the first and second QSTM devices includes a microprocessor and a memory unit operatively coupled therewith, the memory unit storing instructions thereon which, when run on the microprocessor, cause the microprocessor to: detect that the first soft-tissue manipulation is completed and the first QSTM device is in a rest state or placed on its cradle; and perform, based on the detecting that the first soft-tissue manipulation is completed, an automatic self-calibration on the at least one sensor of the first QSTM device before its second use in the same treatment session.

Methods of guiding and analyzing a soft-tissue manipulation treatment performed by one or more practitioners are also disclosed herein. The method includes: providing, by the remote device with an interactive display, a real-time guide for the practitioner during a soft-tissue manipulation treatment session to maintain a target force consistency by setting a target force trendline; recording, by a processing unit, quantifiable metrics associated with a plurality of soft-tissue manipulation stroke types applied using handheld QSTM devices for quantifying the soft-tissue manipulation treatment, the quantifiable metrics being measured by at least one QSTM device associated in a single-device treatment session or a multiple-device treatment session; and generating, based on the quantifiable metrics that are recorded, a composite report of the soft-tissue manipulation treatment involving the QSTM devices for the multiple-device treatment session, wherein the report captures a sequence of treatments in an order of the QSTM devices that are used during the soft-tissue manipulation treatment.

In some examples, the method further includes: classifying the plurality of soft-tissue manipulation stroke types performed by the practitioner using the QSTM devices; determining whether the soft-tissue manipulation stroke types as classified are identical to a plurality of stroke types determined from a history of treatment reports, wherein the history of treatment reports includes force-motion waveform data representing historical soft-tissue manipulation stroke types that are previously recorded; generating a current force-motion waveform data representing the soft-tissue manipulation stroke types and a historical force-motion waveform data representing the historical soft-tissue manipulation stroke types that are considered identical to the soft-tissue manipulation stroke types; and analyzing the current and historical force-motion waveform data to determine a degree of variability between the current and historical force-motion waveform data, wherein the degree of variability is represented as a percentage match.

The term “a” or “an” entity refers to one or more of that entity. As such, the terms “a” (or “an”), “one or more” and “at least one” may be used interchangeably herein. It is also to be noted that the terms “comprising”, “including”, and “having” may be used interchangeably.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a schematic view of a soft tissue manipulation system according to an embodiment of the present disclosure;

FIG. 2A is a 3D representation of a handheld manually operated force-motion applicator device for localized soft tissue manipulation showing treatment plane and device orientation according to embodiments disclosed herein;

FIG. 2B is a 3D representation of a handheld manually operated force-motion applicator device for dispersive soft tissue manipulation showing treatment plane and device orientation according to embodiments disclosed herein;

FIG. 2C is an illustration of a graphical visualization interface for real-time visual feedback of treatment metrics of quantified STM therapy as applied by the practitioner during a treatment session according to embodiments disclosed herein;

FIG. 2D is an illustration of an interactive user interface (similar to an electronic health record) for STM therapy treatment recording, retrieval, comparison and documentation according to embodiments disclosed herein;

FIG. 3 is a schematic view of an electronics assembly of the force-motion application device(s) configured to be used with the soft tissue manipulation system according to an embodiment of the present disclosure;

FIG. 4 is an architectural view of the system highlighting the electronics assembly configured to be used with the soft tissue manipulation system according to an embodiment of the present disclosure;

FIG. 5A is a block diagram of a process indicating the operation modes and working states and their transition actions due to user input as performed by the system during a treatment session according to embodiments disclosed herein;

FIG. 5B is a flowchart of the process of the handheld device for computing and transmitting force-motion data, associated to the STM therapy, to a remote device with interactive display for real-time visual feedback of treatment metrics according to embodiments disclosed herein;

FIGS. 6A and 6B are flowchart diagrams of a process for identifying Stroke count determination of performed STM strokes during therapeutic STM treatment session, by the system according to embodiments disclosed herein;

FIG. 7A indicates the graphical output of the stroke count determination process indicating a true positive peak force of a stroke representing a stroke cycle according to embodiments disclosed herein;

FIGS. 7B and 7C are graphs showing force and motion waveforms obtainable by the system according to embodiments disclosed herein;

FIG. 8 is a graph comparing externally measured forces versus device quantified forces of two devices for quantifying soft tissue manipulation with different maximum force measurement capacities, according to embodiments disclosed herein;

FIG. 9A is a graph indicating angular motion of time versus angle of Drift of Yaw orientation angles calculated according to embodiments disclosed herein;

FIG. 9B is a graph of time versus angle showing orientation convergence by computed sensor measurements according to embodiments disclosed herein;

FIG. 9C is a graph illustrating force validation at a right inclined orientation, according to embodiments disclosed herein;

FIG. 9D is a graph illustrating force validation at left inclined orientation, according to embodiments disclosed herein;

FIG. 10 is a graph of time versus 3D force indicating different treatment sub-sessions combinedly forming a treatment session characterized from the waveforms according to embodiments disclosed herein;

FIG. 11 is a graph of time versus 3D force of different bursts of STM stroke types comprising a treatment sub-session in the form of multimodal dynamic force patterns as measured according to embodiments disclosed herein;

FIG. 12 is a block diagram of the overall process performed by the system to quantify STM treatment according to embodiments disclosed herein;

FIG. 13A is an architectural block diagram of the system setup for quantifying soft tissue manipulation treatments according to an embodiment of the present disclosure;

FIG. 13B is a detailed block diagram of the software architecture reflecting the computing devices associated with the system implementation for quantifying soft tissue manipulation treatments according to an embodiment of the present disclosure;

FIG. 14 is a block diagram of a process performed by the system according to embodiments disclosed herein;

FIGS. 15A and 15B are graphs of time versus force and angular orientation of different directional forces, their RMS, and the associated dynamic roll/pitch/yaw values with the maxima and minima thereof shown according to embodiments disclosed herein;

FIG. 16A is a photograph of an example of a localized force-motion applicator device according to embodiments disclosed herein;

FIG. 16B is a photograph of an example of a dispersive force-motion applicator device according to embodiments disclosed herein;

FIGS. 17A through 17E are photographs of different STM stroke types applied which may be classified by the system according to embodiments disclosed herein;

FIGS. 18A through 18E are graphs of time versus multimodal forces of corresponding STM Stroke types shown in FIGS. 17A through 17E representing individual signature waveform patterns of 3D forces according to embodiments disclosed herein; and

FIGS. 19A and 19B are block diagrams of a process to compare identical classified signature waveform patterns of similarly associated STM stroke types as performed by inter or intra therapist(s) according to embodiments disclosed herein.

It should be understood that the drawings and replicas of the photographs are not necessarily to scale. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Neuromusculoskeletal (NMSK) pain disorders, disease, and injury are leading problems around the globe. NMSK problems escalate alarmingly with aging, producing chronic pain, joint dysfunction or immobility. Chronic NMSK conditions may lead to major surgeries with complicating medications and expensive healthcare visits. Therefore, it is essential to advance non-pharmacological and non-invasive alternatives to traditional medical approaches. Developing nonaddictive, force-based therapeutic modalities that enable quantitative measures to address pain is a high priority for the National Institutes of Health (NIH).

Soft tissue manipulation (STM) is a force-based, non-invasive intervention used to clinically address NMSK pain conditions. It enables a therapist to manually palpate and locate soft tissue restrictions or scar tissues and treat them with externally applied forces in linear or curvilinear fashion, shown to remediate inflammation and enhance blood flow and vascularity. With the current state-of-the-art STM practice, palpation and treatment are performed either by hand only or using tools made of steel or wood for instrument-assisted soft tissue manipulation (IASTM). The penetrable capacity of contoured tooltips, i.e., treatment edges of IASTM tools, can offer resonance-based reverberations to a clinician's hands. This magnifies soft tissue palpation extensively for detecting underlying tissue structures and irregularities. Consistent IASTM on a rodent model proved to enhance healing efficiencies of soft tissue injuries. Human studies with IASTM over a stipulated time also revealed positive implications on the biomechanical properties and neurological behavior of soft tissues. But much remains to be understood about the underlying mechanisms as related to clinical treatment parameters that are needed to achieve optimal outcomes.

Current STM practice standards are mostly subjective suggesting an urgent need for quantitative (or quantifiable) metrics. Current research often uses robotic/mechatronic laboratory setups for mimetic-massage applied on small animals in a uniaxial direction, at targeted area of interest, revealing positive biological outcomes. Additionally, some human studies have applied targeted STM forces, but these methods are either not portable, maneuverable, or durable enough to capture the complex STM force-motions as practically performed by clinicians over multiple areas and body regions. Maintaining targeted pressure consistency along with the motion pattern progression at a reliable pace are fundamental components needed to advance the art of conventional STM. Furthermore, its importance in facilitating students' ability to reproduce an instructor's technique during training is apparent. The lack of scientific rigor to objectively measure STM makes practice reliant mainly on subjective patient-therapist feedback and interactions during treatment. This unrecorded STM is neither adequately documentable nor sufficiently replicable for future reference. This deficiency may devalue the full potential of manual therapy and suggests the urgent need for its characterization with objective, quantitative metrics during realistic STM applications in support of individualized, precision rehabilitation. Quantitative measures are required to better document, monitor, adjust, and progress soft tissue intervention, enable consistent targeted force, capture angular orientation of force application, treatment rate and force-motion pattern progressions for reproducibility in between treatment sessions and users (e.g., clinicians, researchers, instructor-students), and effectively compare results. Therefore, addition of real-time sensory tactile motion feedback to IASTM tools mitigates this deficiency and conceives quantification of soft tissue manipulation (QSTM).

Tactile sensing is common in palpating probes of robot-assisted minimally invasive surgery used for tumor localization or stiffness mapping. Nevertheless, these probes are not designed for adaptive maneuverable therapeutic STM force applicability over wider areas of interest required for treating clinical NMSK conditions. The rate of change of angular force delivery on soft tissue layers over timed intervals using several force-motion signatures in varying paces of application, form the basis of this disclosure and emphasizes the need to quantify STM objectively. Integrating digital technology with IASTM, at least one manually operated novel portable handheld smart medical devices is introduced for evaluating dynamic adaptive continuous real-time localized and/or dispersive force-motions of manual therapy using QSTM.

Embodiments of this disclosure include handheld, portable smart medical devices, for example those disclosed in the U.S. Publication No. 2018/0243158 A1, filed by Indiana University Research and Technology Corp, which is included herein for reference in its entirety, for tracking real-time localized and/or dispersive force-motions to characterize manual therapy treatments as QSTM treatment sessions. In one embodiment a dispersive force-motion applicator includes two 3D load-cells, while in another embodiment a localized force-motion applicator includes a single 3D load cell to quantify compressive and transverse-shear forces of soft tissue manipulation. The load cell(s) of the handheld QSTM device(s) are coupled with a 6 degrees-of-freedom (DOF) Inertial Measuring Unit (IMU) sensor (equipped with 3axis-accelerometer, 3axis-gyroscope, 3axis-magnetometer) for acquiring volitionally adapted therapeutic motions while scanning and mobilizing myofascial restrictions over different areas of the body. These 3D forces measured and the angular orientation of manipulation motions captured characterize QSTM with treatment parameters (targeted force, application angle, rate, direction, treatment motion bursts, motion pattern, time) as a part of post-processing on a computer software (e.g. Q-Ware, intellectual property copyright of which is protected by the Indiana University Board of Trustees). As preliminary findings a human case study was conducted to treat Low Back Pain (LBP) for proof-of-concept of QSTM devices' clinical usability. External validation of treatment parameters reported adequate device precision required for clinical use. The case study findings revealed identifiable therapeutic force-motion patterns within treatments with uniform dose-load (force regimens) leading to subject's elevated force-endurance with self-reported pain reduction by the end of four treatment sessions. As such, QSTM treatment metrics may enable study of STM dosing for optimized pain reduction and functional outcomes using documentable manual therapy. Clinical trials will further determine its reliability and comparison to conventional STM. Therefore this medical device technology is not only aimed at advancing the state-of-the-art manual therapy with precision rehabilitation but also augmenting practice with reproducibility to examine neurobiological responses of individualized STM prescriptions for NMSK pathology.

According to examples disclosed herein, the handheld medical devices may be a localized force-motion sensing QSTM medical device, with a half-disc shaped tapered tooltip specialized for treating smaller regions of interests (digits, wrists, foot/ankle, myofascial trigger points/painful foci, etc.) with shorter massage stroke lengths. This localized QSTM device in conjunction stroke with a dispersive QSTM device, whose operations are supported by a customized clinical computer software (for example Q-Ware), constitutes a comprehensive manual therapy device system needed for patient care. The present disclosure further elaborates the handheld dispersive QSTM device equipped with an elongated convex treatment blade for dynamic force-motion applications as sustained during lengthy stroking treatments over wider and broader surface areas of the body, along with illustrations on a handheld localized QSTM device coupled with a half disc shaped treatment tip for manipulating local or regional areas of the body with shorter stroking treatments. The dispersive handheld device system's architecture is hereby disclosed, as well as working methodology with 3D force and 3D orientation tracking, force-motion gravity correction, and their characterization into QSTM treatment parameters for treatment documentation and replication. The clinical usability of this device system is validated and changes in the soft tissue quality and clinical outcomes discussed in an institutional review board approved case study on a human subject with chronic low back pain (LBP) for proof-of concept. Furthermore, the STM-dose regimen is elaborated in support of the clinical efficacy of using this technology for assessment and treatment of NMSK pain disorders.

For the purposes of promoting an understanding of the principals of the disclosure, reference will now be made to the embodiments illustrated in the drawings, which are described below. The embodiments disclosed below are not intended to be exhaustive or limit the disclosure to the precise form disclosed in the following detailed description. Rather, the embodiments are chosen and described so that others skilled in the art may utilize their teachings. It will be understood that no limitation of the scope of the disclosure is thereby intended. The disclosure includes any alterations and further modifications in the illustrative devices and described methods and further applications of the principles of the disclosure which would normally occur to one skilled in the art to which the disclosure relates.

### System Architecture

Referring to FIG. 1, a quantifiable soft tissue manipulation (QSTM) system 2 includes a QSTM device (force-motion applicator) 4 configured for QSTM or IASTM which is electronically coupled to at least one sensor member 6 and a user interface on a display, illustratively a visual display unit 8. QSTM system 2 is configured to quantify the mechanical load (forces) and manipulation (motion pattern with sustained forces) applied to the soft tissue of a patient during manual therapy in real-time. “Patient” referred to herein may be any human or animal under clinical care and/or any research subject enrolled in research testing, a protocol, a research procedure, etc. More particularly, “real time” refers to immediate quantification and output of data during QSTM such that no delay or a minimal delay (i.e., equal or less than 1 seconds) occurs between measuring the force or pressure and/or angular motion of manipulation applied to the patient's soft tissue and providing the output to visual display 8. For example, soft tissue manipulation system 2 is configured to measure and output the magnitude, rate, duration, and/or angle of the force applied to a handheld device 4 for quantifying soft tissue manipulation by a doctor, therapist, clinician, or other professional to characterize the manual therapy treatment applied to the soft tissue of the patient. The device 4 may be a QSTM device and/or an STM force-motion applicator.

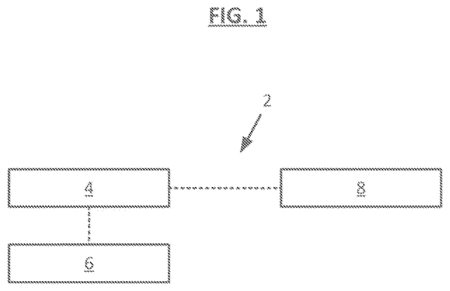

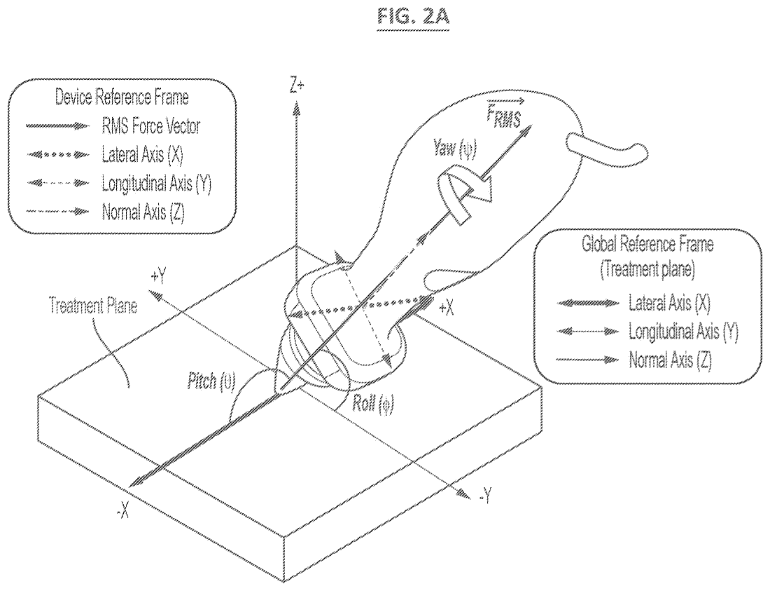

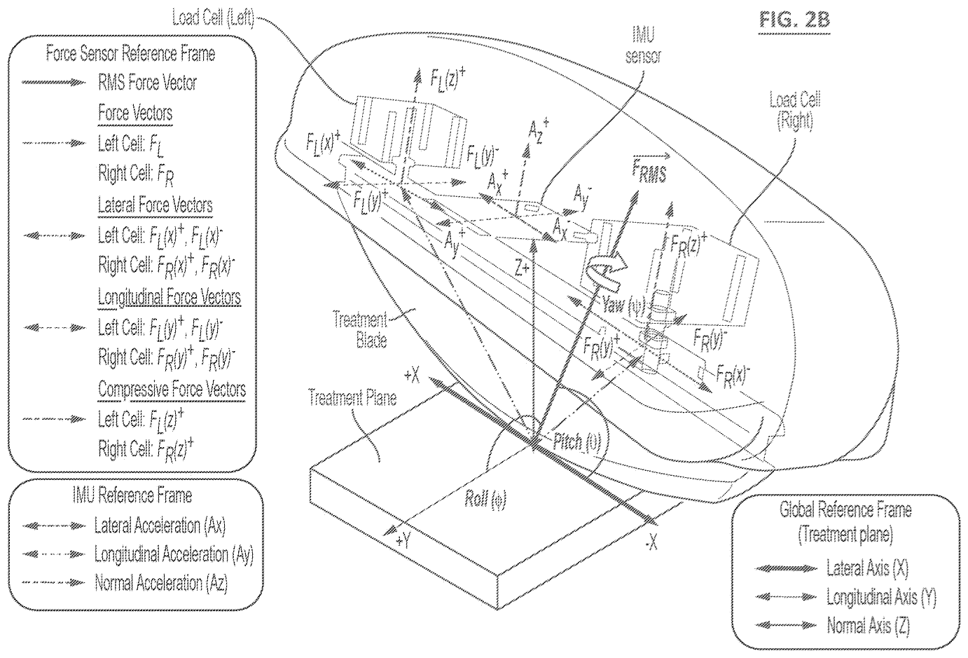

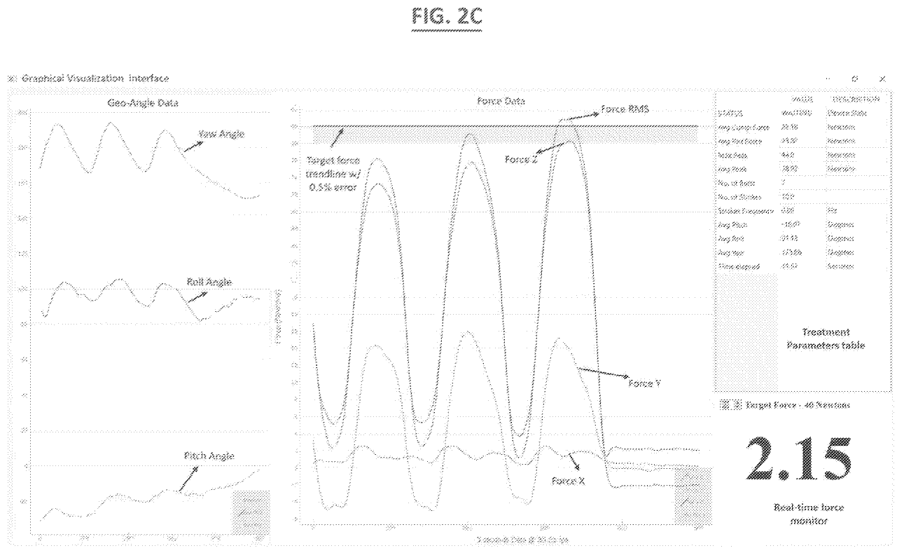

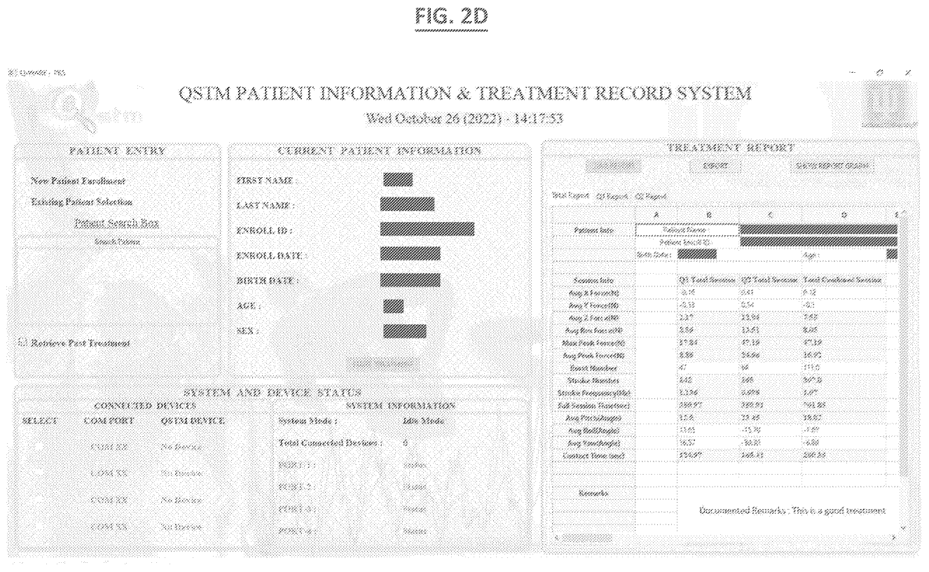

FIG. 2A shows an example of a handheld QSTM device 4, used for localized force-motion based soft tissue massage application on smaller body areas (elbows, knuckles, digits, etc. periphery of bony prominences), which calculates the resultant target force from 3D force and 3D angular orientation information. The device derives the angular motion information, which are essentially yaw (ψ), pitch (θ), and roll (ϕ) angles of the device as shown with respect to gravity direction obtained from sensor fusion of data from IMU motion sensor. IMU sensor data from individual sensing elements such as the accelerometer and/or gyroscope data, for example, are fused by using Quaternion-Euler transformations and/or a non-linear complementary filter-based approach to compute angular orientation of the device with respect to global reference or gravity, a state of art orientation angle computation technique. The figure also shows the device reference frame, including the RMS force vector, shear force components—lateral axis (X), longitudinal axis (Y) along the transverse plane and compressive force component along the normal axis (Z) oriented with respect to the treatment plane aligned to the global reference frame, comprising the transverse lateral axis (X) and longitudinal axis (Y), along with the normal axis (Z). The measured force and motion data is transmitted to the display unit 8 on a remote device, for example a software such as Q-Ware running on a computer with real-time data streaming from handheld QSTM device 4 over a serial communication of 100 Hz using a baud rate of 115.200 kbps. The software performs real-time data visualization on its Graphical Visualization Interface (GVI) and derives necessary treatment parameter(s) such as: compressive force, average resultant force, average treatment angle (device pitch), total treatment time, absolute contact time, average peak force (target force), stroke frequency, etc. to precisely characterize a QSTM treatment with a treatment report for clinical use.

FIG. 2B shows an example of a dispersive handheld QSTM device, angularly oriented with respect to treatment plane indicating rotation angles and force vector transformations from two 3D load cells (“Left” and “Right” as shown, for example). Disclosed vectors and reference frames may include: RMS force vector, individual force vectors of the left and right 3D load cells, comprising shear force components along the lateral and longitudinal force axes of the transverse plane of these load cells, including compressive force components along the normal axis of the load cells. The IMU motion sensor unit is aligned along the load-cell co-ordinate frame shown in FIG. 2B, where lateral acceleration axis (Ax), longitudinal acceleration axis (Ay), normal acceleration axis (Az), are in agreement with the load-cell lateral (X), longitudinal (Y) and normal (Z) axes. The load cell and the IMU sensor together yield the device coordinate frame with respect to the lateral axis (X) of the treatment plane, longitudinal axis (Y) of the treatment plane, and normal axis (Z) of the treatment plane, for example, which is aligned or equivalent to the global reference frame.

Illustrative quantifiable soft tissue manipulation system 2 transmits the force-motion data acquired from sensor member 6 (e.g., a three-dimensional load cell and/or Inertial Measurement Unit—IMU), between device 4 and/or visual display unit 8. The transmission may be performed via a wired or wireless communication.

Additionally, visual display 8 can be positioned at any convenient location that can be viewed by the doctor, therapist, clinician, or professional administering the soft tissue massage to the patient. In one embodiment, visual display unit 8 on a remote device that may be any LED or LCD monitor, display, screen, android or iPhone, Smart TV or other display device configured to display the transmitted QSTM force-motion data from handheld QSTM device 4 in real time.

Alternatively, visual display 8 may be an audio device configured to output a sound to the doctor, clinician, therapist, nurse, or other professional administering QSTM in response to STM stroke types performed by the therapist for individualized patient care. Additionally, visual display 8 may include a combination of both an audio output and a visual output.

A computing device (a remote device for example), including at least memory (such as a non-transitory computer-readable medium) and a processor or microprocessor (processing unit) configured to receive machine-readable instructions or software (for example a multitasking operating system) which may be stored on the memory, is operably coupled to represent a visual display unit. In one embodiment, visual display unit 8 may be a general-purpose multitasking computing device, such as a personal computer (PC), laptop or desktop computer operating on Windows, Linux, macOS or other commercial operating systems; or a smart phone or tablet device running on Android, iOS, Windows, Tizen or other real-time operating systems. As such, sensor member 6 and/or device 4 is configured to transmit sensed data to the computing display device whose processor is configured to convert the sensed data to a treatment report as part of post processing which may be best understood by the user for data visualization and clinical assessment of the therapeutic treatment. For example, the transmitted data may be translated into QSTM treatment metrics for treatment report generation while monitoring graphical feedback of longitudinal force or angular motion along the y-axis, lateral force or angular motion along the x-axis, and vertical force or angular motion along the z-axis normal to the soft tissue of the patient referred as treatment plane in FIG. 2A and FIG. 2B.

The device 4 may be any suitable type of portable, ergonomic, maneuverable under user's volition, mechatronic, and manual (i.e., handheld) soft tissue examination and treatment device. The device may include a treatment end and a handle housing for associated electronic assembly ergonomically designed for clinical convenience. As described herein, the device 4 may be a localized handheld QSTM device that is designed to scan a local area of body (e.g. elbows, knuckles, digits, etc. periphery of bony prominences) for locating soft tissue lesions or fascial restrictions, or a dispersive handheld QSTM device that is designed to scan wider areas of body (e.g., shoulder, upper and lower back, thighs, and lower extremity) respectively, search for tissue irregularities/abnormalities with targeted palpation and treat them with massage based soft tissue force-motion applications as used in the art. Adaptive targeted force-motions using dispersive QSTM device eventually helps release or mobilize such soft tissue abnormalities, promoting blood flow, and attaining painless mobility. The device or force-motion applicator 4 may be coupled with at least one sensor, memory unit, and processing unit to compute and store instructions. A display unit such as visual display 8 has processing and memory capacities to receive and process instructions which are computed into treatment metrics, which may be recorded and permanently stored for immediate post treatment visual observations, future references and analyses. In some examples, the history of recorded treatment metrics on the display 8 may be further analyzed to classify at least one STM stroke type and eventually be used to compare degree of differences (variability) or similarities statistically, numerically, and/or graphically in one or more classified STM stroke types, for example representing individual or identical soft tissue manipulation stroke types performed by the one or more practitioners. In some examples, the comparison may be between treatments performed by the same user (practitioner) or treatments performed by multiple users (practitioners), in order to expand the fidelity of the state-of-the-art manual therapy.

The sensor member 6 may comprise a load cell or force sensor to determine force/dose-load applied to the soft tissue, an IMU motion sensing unit for estimating angular orientation of the device. The load cell may be a force sensing unit including but not limited to a piezoresistive-type or a strain gauge-type sensor. Sensor member 6 is configured to quantify the 3-dimensional shear and compressive forces applied to the soft tissue by handheld QSTM device 4. Moreover, sensor member 6 may comprise a motion sensing unit, essentially a micro-electromechanical inertial measurement sensor or a magnetic, angular rate, and gravity sensor array including but not limited to an accelerometer, gyroscope, magnetometer, which may be integrated inside the handheld housing of the QSTM device 4 to compute the spatiotemporal position or the Angular orientation of the device 4 with respect to global or inertial reference in gravity direction. More particularly, quantifiable soft tissue manipulation system 2 is configured with sensor member 6 to measure and output data with respect to the magnitude (average, maximum, and minimum quantities) of the 3D force applied to the soft tissue with device 4, the duration or time (the duration of overall QSTM procedure as total treatment time, average contact time when device manipulates the soft tissue, and identifies dead times of in between treatment intervals), the frequency or rate (average, maximum, or minimum frequencies) at which a resultant force or does-load is applied to the soft tissue, and the angular orientation at which the manipulation motion proceeds relative to the inertial/global reference frames. These output parameters are transmitted to visual display unit 8 running a software, for example Q-Ware, for real-time visual monitoring and force-motion feedback with response to manipulation force, angle, direction and rate applied to the soft tissue using device 4 as volitionally performed by the practitioner.

In operation, a doctor, therapist, clinician, or practitioner positions device 4 on the soft tissue of a patient at a particular location of an injury or disease. As opposed to automated robotic loading mechanism, or vibrational motions, the doctor, therapist, clinician, or practitioner manually applies a directional pressure on handle of QSTM device 4 which transmits the force through the device's treatment edge on the soft tissue of the patient. This force is transmitted 3-dimensionally deeper into the soft tissue layers in compressive and transverse directions through mechanotransduction to mobilize the neuromusculoskeletal impairment at the treatment site of the human body. The doctor, therapist, clinician, or practitioner in turn feels the reaction forces from the tissue layers as a process of deeper palpation in the form of resonance-based reverberations of the treatment tip or edge which are transmitted to the fingers of the doctor, clinician or practitioner through the device's handle. This haptic feedback from handheld QSTM device 4 magnifies the palpation capacity to assess the underlying structures beneath the skin or subcutaneous layer of the soft tissue while manually guiding the soft tissue manipulation motion on the skin surface. Additionally, the force feedback benefits the doctor to adapt the force sustained motion direction based on stiff inclusions as palpated by the doctor, clinician or practitioner. Furthermore, the real-time sensory feedback from the handheld device is transmitted to the visual display 8 which enhances the user experience and visually monitor the magnitude of force and angular motion applied to the soft tissue to target a particular Force magnitude range and a motion angle for performance continuity of a particular STM Stroke pattern, force-motion signature or portfolio.

The real-time 3D force (Shear X-Y, and Compressive Z components) and the Resultant Force, and the angular orientation of the handheld QSTM device as applied to the soft tissue during manipulation treatment is tracked by the visual display unit 8 in real-time. The visual feedback from display unit 8 enables the doctor, therapist, clinician, or practitioner to monitor the range of applied force and adjust the magnitude or direction based on the readings on the visual display to maintain consistency. For example, the real-time data stream from the handheld QSTM device 4 may be displayed to the doctor, therapist, clinician, or professional on visual display unit 8 contained in a remote device (e.g. a Personal Computer), running the Graphical Visual Interface (GVI) as shown in FIG. 2C of Q-Ware (a PC software), such that force components (lateral X, longitudinal Y, compressive Z) and the angular device orientation from yaw, pitch, and roll measurements of a particular motion pattern applied to the soft tissue by device 4 is outputted in form of color-coded graphical waveforms or black-and-white graphical waveforms, as appropriate. Each waveform may be generated using multimodal waveform pattern data as measured by the sensor member 6. Alternatively, GVI on visual display unit may portray this data on a multi-visualization window comprising a visual digital scale (numeric force monitor); live or animated color-coded multichannel charts for visualizing the real-time force and angular orientation waveforms; and a treatment parameters table to display numeric magnitudes (averages and maximum) of force and angular motion components, the frequency or rate of treatment, number of strokes applied and the time taken to perform a particular stroke pattern or a force-motion sequence as performed by the doctor, clinician or practitioner on the soft tissue of the patient. The live animated chart(s) may display color-coded graphical force and angular motion waveforms with respect to time duration, such that the charts are user customizable and minimizable. These color coded charts may represent 3D Force with a first color (e.g., red) indicating lateral force (X), a second color (e.g., green) indicating the longitudinal force (Y), and a third color (e.g., blue) indicating the compressive force (Z), and fourth color (e.g., brown) indicating resultant (Root Mean Squared) force value. Similarly, the color-coded charts representing Angular Orientation of the device may display temporal waveforms of geometric-yaw angle in red color indicating device's side to side movement, geometric-pitch angle in green color indicating back and forth movement and geometric-roll angle in blue indicating the rotation along the axial direction of the device, thus completing the measurements of an Attitude-heading-reference-system representation. Additionally, a user defined “target force trendline” with 0.5% error can be set on the force-chart to monitor the targeted force peak of the resultant force per STM stroke (force-motion cycle) applied to the soft tissue. The status of the device is also visualized in the GVI of the software. Visual monitoring of applied force magnitudes from force charts and uniform maintenance of the maximum force peaks per stroke cycle within a percent of the set target force may improve consistency of the doctor, clinician or practitioner to minimize intra-practice variabilities. The live data stream as visualized on the visual display unit may be translated into treatment reports for clinical use and stored with documented comments in a local database at the permanent memory of the visual display unit 8 for later review, future referencing comparison and analysis or exporting to an associated electronic health record. In this way, soft tissue manipulation system 2 can be used to provide consistent and replicable pressure to the patient during a massage-based therapy over the course of a treatment or therapy schedule. Additionally, guidelines, standards, and/or best practices can be created about the type of pressure which is most effective for particular injuries and/or diseases because real time force quantification is possible with soft tissue manipulation system 2.

In one embodiment FIG. 2D show an example of the patient-treatment record system (PRS) of the QSTM software (Q-Ware for example) which is analogous to an electronic health record. The PRS represents information of the current patient or subject being treated including information regarding the patient such as the name, ID number, enrolled date, birth date, age, sex, and any other suitable information for patient identification. Also included, as shown in FIG. 2D, may be the Treatment report comprising session information for multiple sub-sessions of the treatment, including values such as average forces (X, Y, Z, and resultant), maximum peak force, average peak force, burst number, stroke number, stroke frequency, full session time, average pitch/roll/yaw, contact time, etc. Also, remarks may be displayed regarding the session, for example whether the session or treatment was good (satisfactory) or bad (unsatisfactory), including anatomical notes about the underlying structures of the soft tissue under treatment along with the protocol assumed for treatment as suitable. The PRS also offers to display the status of QSTM device connected to the display unit.

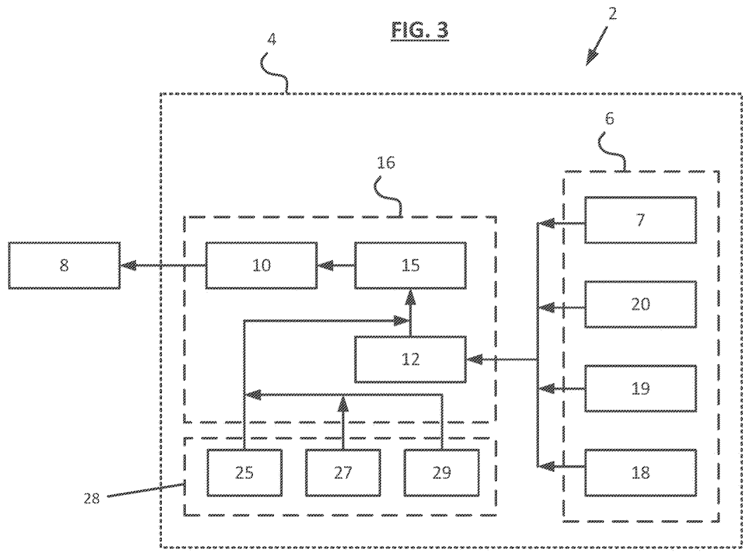

Any of handheld QSTM devices 4 may be configured to operate with any suitable electronics assembly, with respect to FIG. 3, where the electronics assembly is operably coupled to and/or supported by handheld QSTM device 4. More particularly, sensor member 6 may be supported within or house inside the handheld QSTM device 4. Additionally, device 4 also may support a microcontroller unit 16 operably coupled to visual display 8. The microcontroller unit 16 may comprise processing unit 15 operably coupled to an inbuilt data acquisition unit 12 for converting analog force or angular motion values sensed as electrical voltages into machine readable standard units, and a receiver/transmitter 10 for serial communication of output data-stream to display unit 8.

Electronics assembly of handheld QSTM device 4 also includes peripheral electronics unit 28 such as which may contain a control button 25, an LED (e.g., RGB LED lights) 27 for visualizing operation states of the handheld QSTM device 4, and a memory unit 29 (e.g. an SD card) which is operably coupled to the microcontroller 16. More particularly, data acquisition unit 12 operably couples measurements from sensor member 6 which may include data from 3D Force sensor member 7 in shear (lateral and longitudinal) and compressive directions, data from tri-axial accelerometer 20 to extract linear 3 dimensional accelerations and accelerations due to gravity, angular rotational velocity measurements from a tri-axial gyroscope 18 and magnetic field measurements (e.g. a compass) with respect to earth's geomagnetic effects from a triaxial magnetometer 19. The data from these sensor members are read by the data acquisition unit 12 in the form of electrical voltages and sampled, filtered, and processed by the processing unit 15 into mathematical units of force in newtons and angles in degrees or radians, as used in the art of mathematical sciences. Such computed quantities from the processing unit are sent to the receiver/transmitter 10 to package as data packets for wired or wireless transmission protocol. The packaged data are transmitted from the microcontroller 16 at a baud rate (number of data packets delivered per second) to the visual display unit 8. The display unit visualizes the data in color-coded graphical waveforms for real-time visual monitoring of treatment metrics to minimize practice inconsistency, improve treatment approaches, and document quantified treatment for clinical use, treatment assessment and future referencing.

During a therapy session or appointment, a doctor, nurse, therapist, or other professional ensures QSTM device 4 is powered on and/or otherwise actively connected to electronics assembly through a wired or wireless connection. The doctor, nurse, therapist, or other professional then contacts the patient's soft tissue with the handheld force-motion applicator or device 4 and applies force on the handle of device 4 along a manipulation motion direction which is then transferred to the patient's soft tissue through the treatment edge of the handheld QSTM device 4. The real-time force-motion data is tracked as graphical waveforms and subsequent treatment reposts are generated with treatment parameters (e.g. Average Resultant force, Target force, Average angular orientation—yaw-pitch-roll combinations, number of strokes applied, number of bursts applied, average manipulation frequency, Total treatment time duration, average device to skin contact time duration, treatment interval time duration, etc.). As such, the Force and Motion data may be recorded as raw treatment file while the individual treatment reports for each QSTM device used, or combination of all QSTM devices used in every treatment session performed may be recorded and stored within computing device of the display unit 8, for example in a permanent memory of the computing device associated with visual display 8, such that a patient's treatment record, therapy log or plan, or other medical notes may be updated and retrieved for subsequent appointments with the patient. This treatment record of treatment approaches (Manipulation motions maneuvered by different clinician, practitioner or doctor) enables comparison of treatment patterns, determine inconsistencies or variabilities in applied STM strokes both intra and inter therapists and offers education protocols for practice and reproducible therapy procedures for the same dose-load or force-motion regimen used by expert clinician or therapist.

In one embodiment, visual display 8 may be configured to allow a doctor, nurse, therapist, clinician, or other professional to start at a single location on the patient's soft tissue and move force-motion applicator along a certain direction following a motion progression of a distinguished STM stroke type as used in manual therapy art. The combination of waveform patterns of individual force and motion components for every stroke (force-motion cycle or repetition) can be harnessed to classify distinctively identified stroke types. The underlying soft tissue irregularities (stiff inclusions, bands or nodules) which are palpated during directed gliding motion of the handheld QSTM device during therapeutic manipulation is very evident from the graphical waveforms. For example, healthy soft tissue may feel like smooth sheets of paper such that device 4 easily glides over the soft tissue. Conversely, unhealthy, damaged, or aged soft tissue may feel like crumpled paper such that force-motion applicator records bumps, creases, or other uneven tone or surface dimensions of the soft tissue structure. These irregularities due to the underlying unevenness of the soft tissue are well captured in the graphical waveforms of the force-motion visualization data on the visual display unit 8 in the form of discontinuities in the temporal force graphs. These discontinuities can be studied more to assess the condition of the soft tissue. In this way, soft tissue manipulation system 2 is configured to provide digital feedback of the manipulation and maneuvers of the practitioner during soft tissue treatment to assess soft tissue irregularities or unevenness, thus characterizing soft tissue health.

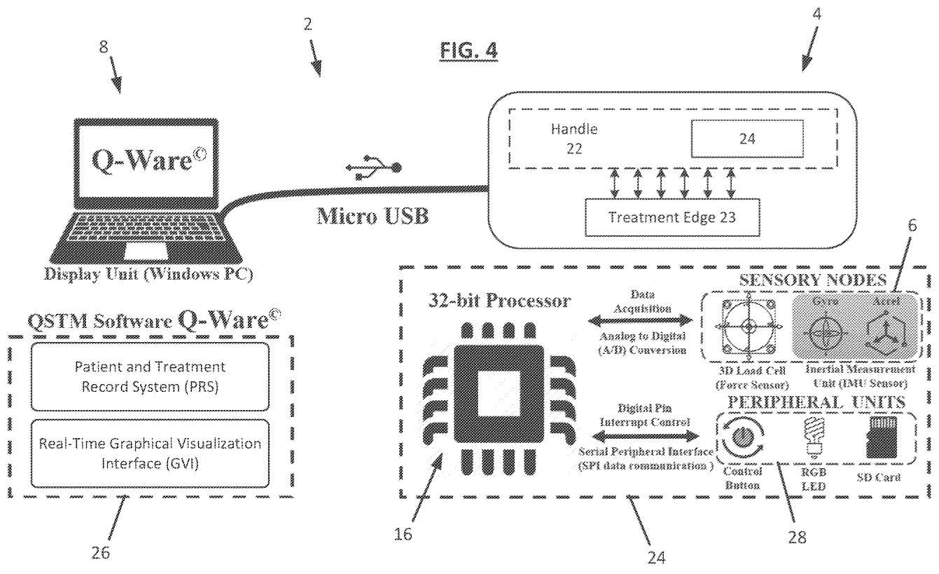

FIG. 4 shows an exemplary embodiment of the soft tissue manipulation system 2 as disclosed herein. The system 2 includes the device 4 which includes a plurality of device hardware components 24 such as the aforementioned sensors 6 and microcontroller 16, a force-motion applicator with a handheld part or handle 22 of the device 4 housing the electronic hardware components 24, wherein the handle 22 is mechanically coupled with a treatment edge 23 for sensing reaction forces of the dose-load applied to the soft tissue while positioning the QSTM device 4 onto the soft tissue site or treatment plane for massage and tissue manipulation as explained above, and the aforementioned visual display 8 coupled with software components 26 which may include one or more computing device such as processor as mentioned above. The device 4 may be coupled with the visual display 8 (or a computing device which is coupled with or includes the visual display 8) via a wire or cable such as a Micro USB cable or wirelessly such as via infrared, Bluetooth, Wi-Fi, Zigbee, ultra-wide band (UWB), or any other suitable means known in the art. The visual display 8 may be a display unit of a computer, on which is installed the suitable software for QSTM such as Q-Ware, and the software components 26 may include Patient-Treatment Record System (PRS) and real-time Graphical Visualization Interface (GVI) which operate to provide the graphical output as explained above. The hardware connectivity of electronics components 24 may further include peripheral units 28 with a control button 25, RGB-LED 27, memory unit 29 operably coupled with a microcontroller 16 with processing unit 15, data acquisition unit 12, and receiver/transmission unit 10. Force and motion computation of the STM dose-load (force) and manipulation angular motion of the QSTM device 4 as maneuvered by the practitioner during treatment may be performed using any suitable methods or processes as disclosed herein. The microcontroller 16 may be a 32-bit processor (or any other suitable type of processing unit as known in the art) operatively coupled with the sensors 6 (e.g. 3D load cell or force sensor, and inertial measurement unit such as the gyroscope and accelerometer) via analog-to-digital (A/D) data acquisition link, and operatively coupled with the peripheral units 28 via digital plan interrupt control link such as serial peripheral interface such as SPI data communication link.

The computations of the system 2 may be distributed between an embedded firmware on the microcontroller 16 in the device and a computer Software (Q-Ware) developed to operate multiple QSTM medical devices for clinical use. The embedded firmware is a multithreaded application. It performs sensor-data acquisition, device calibration, force quantification, device tilt sensing, and serial communication to the computer. Additionally, the firmware executes control button-based interrupt service routines for switching operational states during the treatment mode of the device 4. The six axes' measurements from two 3D load cells are transformed into three force components i.e., compressive (vertical-Z) normal to the treatment plane and planar (shear-X and tensile-Y) along the lateral and longitudinal direction of the blade's point of contact (see “Treatment Blade” of FIG. 2B, for example). The average magnitude of the RMS Force vector forms the resultant Dose-Load of every force-motion stroke cycle applied during treatment, as shown in FIG. 2B. Whereas Q-Ware, operating on a computer, being a multiprocessing software executes multiple tasks. It features a local PRS for patient treatment data management; a device-specific GVI for real-time 3D force and motion tracking and data monitoring; and a Data Analysis Interface for visual comparison or assessment of several force-motion portfolios applied during treatment. In addition, the PRS for importing recorded data is incorporated for referencing and assessment of patient progress.

### Methodology

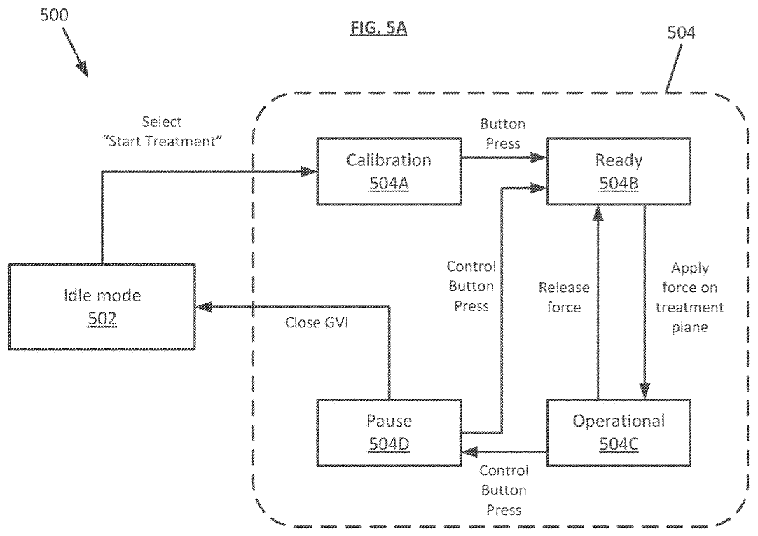

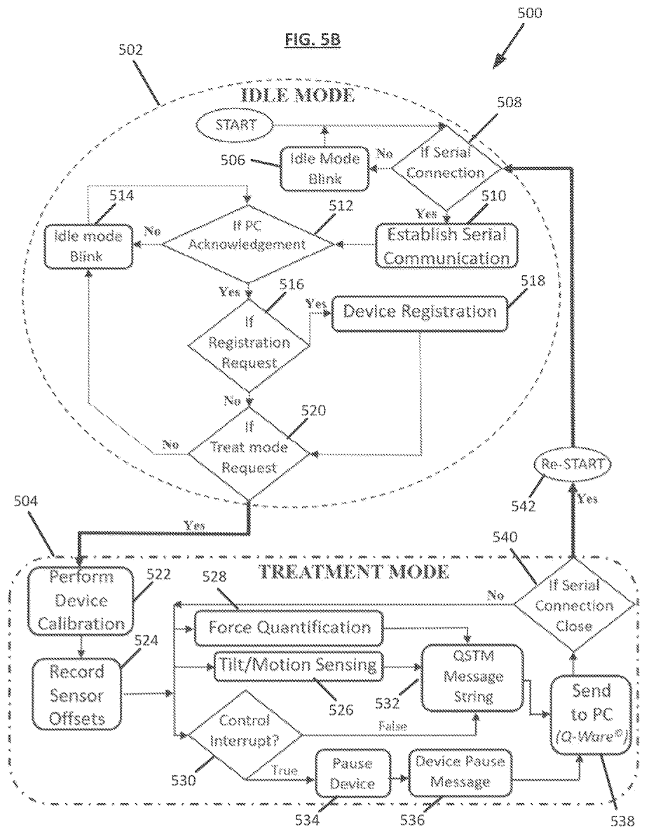

FIG. 5A shows a simplified block diagram of a process 500 which may be performed by the system 2 according to embodiments disclosed herein. The process 500 includes two (2) system modes: Idle mode 502 and Treatment mode 504. The Treatment mode 504 has four (4) states: calibration 504A, ready 504B, operational 504C, and pause 504D. The workflow of the tasks performed by the handheld device 4 in its system modes 502 and 504 is explained. In Idle mode 502, the device first establishes serial communication with the computer, registers itself on the software, e.g. Q-Ware, with identification information, and then waits for user requests to start Treatment mode 504. This mode may be indicated by a 1 Hz white LED blink on the device 4, while resting on its cradle in a predefined position, as shown in step 506 of FIG. 5B, which shows a more detailed block diagram of the process 500.

Specifically, in step 508, the device determines if the serial connection is established. If there is no connection, the device returns to step 506, i.e. perform the idle mode blink. If there is established connection, as determined or confirmed in step 510, the device proceeds to step 512 to determine if the computer (e.g., PC) acknowledges the device. If there is no acknowledgement, the device proceeds to idle mode blink in step 514; otherwise the device proceeds to step 516 in which the device determines if registration request is received. If so, in step 518, the device proceeds to perform registration, then proceeds to step 520 to determine if Treatment mode request is received; otherwise, the device proceeds from step 516 directly to step 520. In step 520, if no request is received to enter Treatment mode, the device returns to step 514; otherwise, it proceeds to step 522 in the Treatment mode 504.

The Treatment mode 504 starts with user's “start treatment” selection on Q-Ware, with a solid red LED glow, indicating the calibration state 504A, which is shown as step 522 in which device calibration is being performed. The device 4 should be left untouched during calibration, until a solid green LED glow indicates calibration is complete. After calibration in step 522, a control button press on the device starts the ready state 504B which initiates step 524 to record sensor offsets. The Treatment mode 504 performs a multithreaded operation of three tasks: tilt orientation sensing with respect to gravity (step 526), 3D force quantification (step 528), and executing Interrupt Service Routines (ISR) based on control button input (step 530). A solid blue LED glow indicates both the ready and operational state 504C of treatment mode, while the device pause state 504D, triggered by a device button press, is indicated by alternate white and pink led blinks every second.

Explanation is provided below with regard to the tilt orientation sensing with respect to gravity according to step 526 according to some embodiments. The instantaneous device orientation angle is essential for determining the force application angle with respect to the gravity/global frame of reference. The 16-bit 3D accelerometer and 3D gyroscope data, acquired from the IMU sensor, are transmitted through 12C communication protocol to the microprocessor. The 3D acceleration and 3D gyro biases are eliminated using bias offsets determined, in step 524. These 6 DOF data are fused at 200 Hz sampling rate to estimate precise orientation angles.

A complementary filter-based approach may be initially implemented to find optimal location for positioning IMU sensor on the device. The IMU sensor may be placed at the center of the handheld device and aligned with the 3D load cell lateral axis to avoid orientation mismatch, as shown in FIG. 2B. The filter-based approach may be updated with quaternion transformations as known in the art, using any modified gradient descent-based Attitude Heading Reference System filter as suitable. Solving the gimbal lock situation and minimizing computation overhead are the major advantages of using quaternions over Euler angle-based computation. The filter uses a unit gain parameter (β=1) and is updated at a frequency of 200 Hz. The filter output yields a four-element unit quaternion vector explained in Equations (1) and (2) below.

$${Q}_{o} + {Q}_{1} i + {Q}_{2} j + {Q}_{3} k \qquad ( 1 )$$

In Equation (1), Qₒ is the scalar quantity describing the rotation angle and Q₁, Q₂, and Q₃ are coefficients of axis-vector components describing the orientation in Euclidean space (i, j, k). So essentially, if a unit vector axis of rotation [x, y, z], is rotated by an angle α, then the quaternion for this rotation will be of the form shown in Equation (2):

$$cos ( \frac{α}{2} ) + sin ( \frac{α}{2} ) ( xi + yj + zk ) \qquad ( 2 )$$

where the norm of all four components will be equal to 1. The elements of the unit quaternion are further transformed into Euler angles in degrees of the form Yaw (ψ), Pitch (θ) and Roll (Ø); which are rotations about the Z, Y and X axes of the handheld device in global coordinate frame respectively. Equations (3) through (5) below explain the quaternion to Euler angle conversions to estimate real-time orientation angles of the device:

$$ψ = {tan}^{- 1} ( \frac{2 ( {Q}_{1} {Q}_{2} + {Q}_{0} {Q}_{3} )}{{{Q}_{0}}^{2} + {{Q}_{1}}^{2} - {{Q}_{2}}^{2} - {{Q}_{3}}^{2}} ) \times \frac{180 °}{π} \qquad ( 3 )$$

$$θ = {sin}^{- 1} ( - 2 ( {Q}_{1} {Q}_{3} - {Q}_{0} {Q}_{2} ) ) \times \frac{180 °}{π} \qquad ( 4 )$$

$$∅ = {tan}^{- 1} ( \frac{2 ( {Q}_{0} {Q}_{1} + {Q}_{2} {Q}_{3} )}{{{Q}_{0}}^{2} - {{Q}_{1}}^{2} - {{Q}_{2}}^{2} + {{Q}_{3}}^{2}} ) \times \frac{180 °}{π} \qquad ( 5 )$$

The output of this filter provides a fast response with almost no visualized lag of angular orientations minimizing latency and jitter. The gain parameter is tuned to match the gyro bias for integration drift compensation and improve steady and random dynamic motion sensing accuracy.

Explanation is provided below with regard to the 3D force quantification according to step 528 according to some embodiments. The step includes the voltage signal acquisition of 3D forces and conditioning in Equation (6), and the transformation of the acquired voltage signal to force units (Newton) in Equations (7) through (9) using the calculated offsets and calibration parameters, and the obtained resultant force is further corrected to minimize the gravity effect of blade's weight by kinematic transformations in Equations (12) through (15) using rotation sequence in Equations (10) and (11), as explained below.

The analog output from 3D load cells, sampled at 500 Hz with 16 bit resolution are recorded for offset voltage determination at the calibration step. The force offset voltage vector (\vec{V_{off}}) for both load cells are obtained by the measured mean of each of the six channels' thermal noise distribution calculated during calibration (no-load condition of device). The difference of load-voltage vector (\vec{V})ⱼ, with dimension [x, y, z], from offset voltage vector \vec{V_{off}} for each load cell, at the j^{th} iteration, is fed into a rolling mean filter of sample size (n=25) for removing high-frequency channel noise.

$$\vec{{V}_{F}} = \frac{1}{n} {\sum }_{j = 0}^{n - 1} {( \vec{V} )}_{j} - \vec{{V}_{off}} \qquad ( 6 )$$

The matrix representation of force-signal vector \vec{V_{F}} from Equation (6) above is transformed into an actual force vector \vec{F} in Newtons, by multiplying with (voltage-force) characterization matrix A_{3×3}, given by the load-cell manufacturer. The blade, being suspended from sensors exerts a tension force due to its weight along gravity direction. This causes an offset voltage baseline shift of ξ volts along the F_{Z} axis, aligned to gravity, at calibration. As a result, the device suffers force measurement skewness at other orthogonal axes when it is rotated in different orientations. The orthogonal force measurement skewness is resolved using a voltage correction, by subtracting a offset voltage calibration error (ξ volts) along gravity aligned to sensor axis at calibration orientation. This correction, shown in Equation (7) below, ensures uniform orthogonal distribution of blade's weight along the device's local coordinate system.

$$[ \begin{matrix}{F}_{x} \\ {F}_{y} \\ {F}_{z}\end{matrix} ] = [ \begin{matrix}{A}_{11} & {A}_{12} & {A}_{13} \\ {A}_{21} & {A}_{22} & {A}_{23} \\ {A}_{31} & {A}_{32} & {A}_{33}\end{matrix} ] \times [ \begin{matrix}{V}_{F ( x )} \\ {V}_{F ( y )} \\ {V}_{F ( z )}\end{matrix} ] - [ \begin{matrix}1 & 0 & 0 \\ 0 & 1 & 0 \\ 0 & 0 & {A}_{33}\end{matrix} ] \times [ \begin{matrix}0 \\ 0 \\ ξ\end{matrix} ] \qquad ( 7 )$$

Equation (7) is deployed for voltage-force characterization and orthogonal force corrections of both left and right load cells. The individual force components of the respective left and right load cells are added up to yield a 3D force vector as shown in Equation (8):

$$[ \begin{matrix}{F}_{3 D ( x )} \\ {F}_{3 D ( y )} \\ {F}_{3 D ( z )}\end{matrix} ] = {[ \begin{matrix}{F}_{x} \\ {F}_{y} \\ {F}_{z}\end{matrix} ]}_{L} + {[ \begin{matrix}{F}_{x} \\ {F}_{y} \\ {F}_{z}\end{matrix} ]}_{R} \qquad ( 8 )$$

The 3D force components in Equation (8) are root mean squared to achieve the resultant force of the handheld device.

$${F}_{RMS} = \sqrt{{( {F}_{3 D ( x )} )}^{2} + {( {F}_{3 D ( Y )} )}^{2} + {( {F}_{3 D ( Z )} )}^{2}} \qquad ( 9 )$$

F_{RMS} produces the resultant instantaneous force along the moving force co-ordinate system F_{B} of the handheld device, where B represents the local reference frame of the device. The orthogonally distributed weight of blade adds tension forces along gravity direction when the device is rotated without applying forces. These tension forces present in the moving force coordinate system F_{B} needs gravity correction. This correction is facilitated by the transformation of measured 3D forces from local device co-ordinate frame B into the global inertial co-ordinate frame I, by forward kinematic equations. Here, F_{I} denotes the inertial force co-ordinate system, where all relative accelerations are assumed to be zero. A 3D rotation transformation matrix R_{(ψ,θ,Ø)}^{3×3}, is derived in Equations (10) and (11) using the combination of rotation angles from Equation (3), Equations (4) and (5) with Euler rotation combination sequence of ZYX axes:

$${R}_{( ψ , θ , ∅ )}^{3 \times 3} = {Rot}_{Z} ( ψ ) \times {{Rot}_{Y} ( θ )}^{T} \times {Rot}_{X} ( ∅ ) \qquad ( 10 )$$

$${R}_{( ψ , θ , ∅ )}^{3 \times 3} = [ \begin{matrix}{C}_{ψ} {C}_{θ} & {C}_{ψ} {S}_{θ} {S}_{∅} - {S}_{ψ} {C}_{θ} & {C}_{ψ} {S}_{θ} {C}_{∅} + {S}_{ψ} {S}_{∅} \\ {C}_{ψ} {C}_{θ} & {C}_{ψ} {C}_{∅} - {S}_{ψ} {S}_{θ} {S}_{∅} & - {C}_{ψ} {S}_{∅} - {S}_{ψ} {S}_{θ} {C}_{∅} \\ {S}_{θ} & {C}_{θ} {S}_{∅} & {C}_{θ} {C}_{∅}\end{matrix} ] \qquad ( 11 )$$

In these equations, Rot_{Z}(ψ), Rot_{Y}(θ), and Rot_{X}(Ø) are the rotations about Z, Y, and X axes, while C_{ψ}=Cos(ψ) and S_{ψ}=Sin(ψ) and corresponding sines and cosines of the other rotation angles. Now applying forward kinematics, the 3D force vector from Equation (8) is converted from local to the inertial co-ordinate system using 3D transformation matrix from Equation (11).

$${F}_{I ( x , y , z )}^{B ( 3 \times 1 )} = {R}_{( ψ , θ , ∅ )}^{3 \times 3} = \times {[ \begin{matrix}{F}_{3 D ( x )} & {F}_{3 D ( y )} & {F}_{3 D ( z )}\end{matrix} ]}^{T} \qquad ( 12 )$$

In the above equation, F_{I}^{B} represents the force transformation from local frame B to inertial frame I. Since the blade's absolute weight (M_{b} Newtons) is aligned along the F_{Z} direction during initial calibration, it needs to be subtracted from the inertial frame's Z component of the Force vector to eliminate the additive forces due to blade's weight.

$${F}_{{I ( x , y , z )}_{updated}}^{B ( 3 \times 1 )} = {F}_{I ( x , y , z )}^{B ( 3 \times 1 )} - {[ \begin{matrix}0 & 0 & {M}_{b}\end{matrix} ]}^{T} \qquad ( 13 )$$

Finally, the updated inertial forces in Equation (13) are transformed back to the moving force co-ordinate system of local reference frame by applying inverse kinematic transformation of the rotation matrix from Equation (11) as shown in Equation (14).

$${F}_{{B ( x , y , z )}_{updated}}^{I ( 3 \times 1 )} = {[ {R}_{( ψ , θ , ∅ )}^{3 \times 3} ]}^{- 1} \times {F}_{{I ( x , y , z )}_{updated}}^{B ( 3 \times 1 )} \qquad ( 14 )$$

The updated forces yield error diminished weight corrected measurements in all orientations, where the force noise levels at each axis are confined to 0.2 Newtons.

$${F}_{B ( RMS )}^{I} = \sqrt{{( {F}_{B ( x )}^{I} )}^{2} + {( {F}_{B ( y )}^{I} )}^{2} + {( {F}_{B ( z )}^{I} )}^{2}} \qquad ( 15 )$$

Hence the resultant force F_{B(RMS)}^{I} in Equation (15) forms the instantaneous dose-load of STM at every force-motion stroke cycle during a clinical treatment session.

After each of steps 526 and 528 is completed, the resulting data is transformed into a QSTM message string in step 532. Additionally, the device may determine if there is any control interrupt in step 530 simultaneously with performing the aforementioned tilt/motion sensing step 526 and force quantification step 528, and if there is no control interrupt detected, the device likewise proceeds to step 532. If there is a control interrupt detected, the device proceeds to step 534 where the device is paused, and then to step 536 in which the device pause message is generated. Both the QSTM message string from step 532 and the device pause message from step 536 are then sent to the computer running the software (Q-Ware) in step 538, after which the device determines if serial connection (or any suitable data connection as known in the art) with the computer has been closed, in step 540. If the connection is not closed, the device repeats the process by simultaneously performing steps 526, 528, and 530 as previously explained; otherwise, the device restarts in step 542 and returns to the aforementioned step 508 in the Idle mode 502.

In some examples, the magnitude of the resultant force F_{B(RMS)}^{I} from the force quantification step 528 may be harnessed to find a threshold for determining whether the device is in contact with skin or not. The treatment blade weighs 2.5N (˜250 grams). Jerks or swift rotations might trigger sudden force due to inertial momentum. Henceforth the threshold magnitude is set to 1 Newton (much greater than the force noise level). Resultant forces above the threshold determine the operational state of treatment mode, while that below threshold indicates a ready state (device waiting to be used). The control button is used to switch the device from the Operational state to Pause state during treatment mode by an alternate button press. The Pause state is marked by an alternate pink and white LED blink. The sum of the time accounted for both Ready and Pause states of the device defines the dead time of the entire session.

In some examples, the QSTM message string generated in step 532 comprises of the 3D Force Vector [F_{B(x)}^{I}, F_{B(y)}^{I}, F_{B(z)}^{I}]^{T}, the Resultant Force F_{B(RMS)}^{I}, the geo-orientation angles yaw (ψ), pitch (θ) and roll (Ø) with respect to gravity, acceleration, and gyroscope vectors from IMU, along with the control button state (High/Low). This string is sent to the computer's software (Q-Ware) at a serial transmission frequency of 100 Hz with a USB baud rate of 115.200 kbps. The quantified force-motions data delivered to Q-Ware is processed to yield QSTM Treatment parameters. These parameters include average compressive force, average resultant force, maximum peak force (maximum of all local maxima in the resultant force stream), target force (average of all peak forces of all force-motion cycles during a treatment session matched with a user defined target), number of treatment strokes, skin-contact time, elapsed treatment time and stroke frequency.

The computer running the software (Q-Ware) may streams and display the force and motion data on its Graphical Visualization Interface (GVI) for real-time visualization using a time-division multiplexing algorithm at a variable framerate. It also saves the raw data stream in a csv file for post-processing, future referencing, and analysis. The resultant force F_{RMS} stream is first subjected to a sliding window Low Pass Filter (LPF), and then searched for local maxima and minima to generate force peak-valley pairs.

Noise filtering may be also performed according to the following method. A digital low pass filter with a discreet binomial kernel, derived from the binomial distribution, of the form shown in Equation (16):

$${F}_{RMS ( LPF )} = {\sum }_{k = 0}^{n} ( ( \begin{matrix}n \\ k\end{matrix} ) \times \frac{\sqrt{n}}{{2}^{n}} \times {F}_{RMS} ) / {\sum }_{k = 0}^{n} ( ( \begin{matrix}n \\ k\end{matrix} ) \times \frac{\sqrt{n}}{{2}^{n}} ) \qquad ( 16 )$$

may be implemented to smooth noise frequencies of the original signal; where n is window size and k is the window iterator. Another higher order (N=10) Butterworth filter using a cut-off frequency of 11 Hz was implemented to match the results of Binomial Kernel based LPF with window size (n=25). The latter performs better for steady motions as compared to the former which achieves better signal to noise ratio for nondeterministic sporadic motions. Hence, there is a tradeoff in smoothing out noise due to hand vibrations during force application and retaining essential signal ripples observed due to tissue irregularities (tight spots, nodules) of the underlying skin contour.

Furthermore, treatment stroke determination may be performed according to the following method. Each Treatment Stroke may be determined by the maximum resultant peak force per force-motion cycle, discarding the redundant peaks (due to hand vibrations/tissue irregularity) from stroke count consideration for every force-motion portfolio. Therefore, a decision tree-based algorithm is designed to eliminate redundant peaks and detect maximum force peak per cycle for stroke identification and treatment rate estimation.

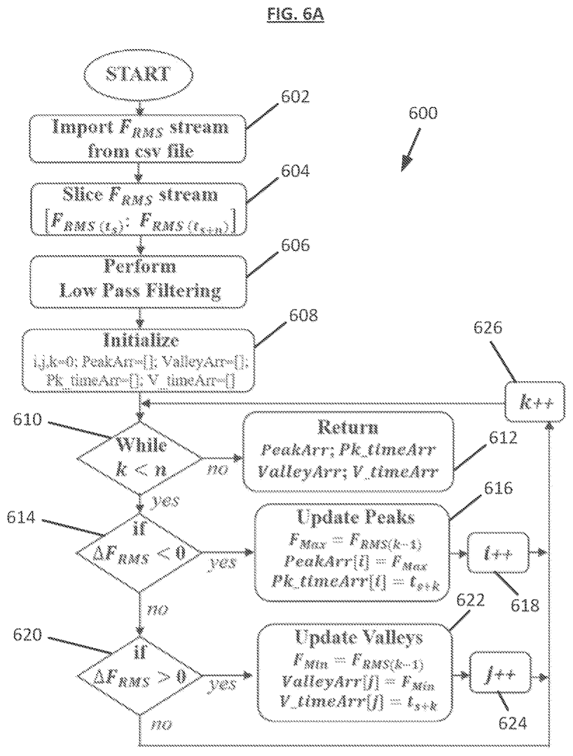

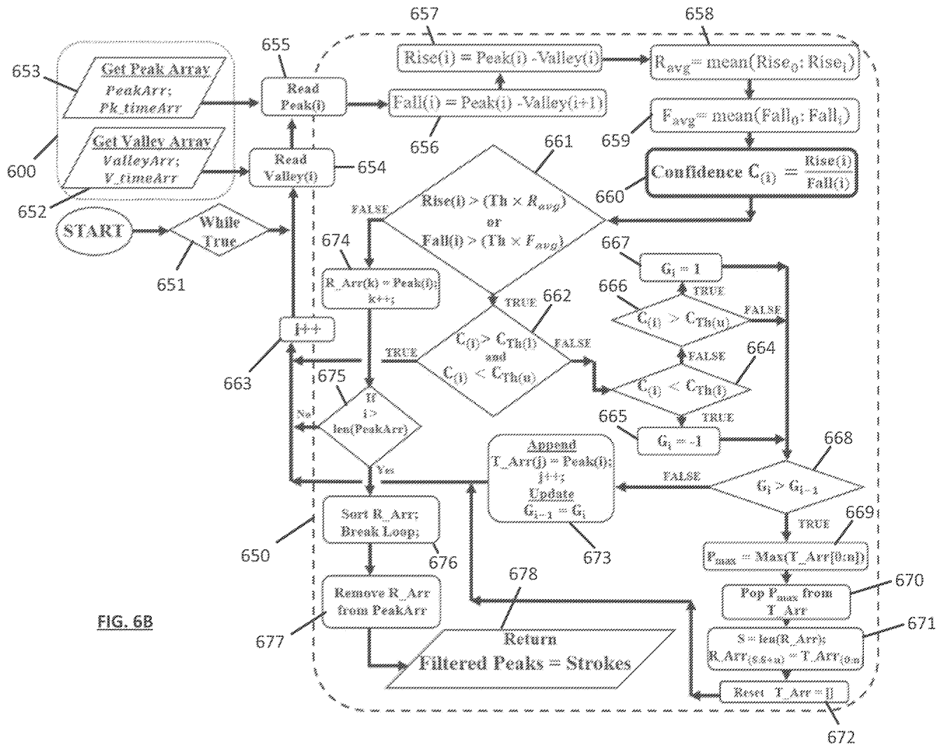

Decision tree-based treatment rate estimation may be performed according to the following method. The peak-valley pairs are generated from the gradients of the real-time filtered force data stream using the algorithm or process described in flowchart of FIG. 6A. These peak-valley pairs form the fundamental features for the decision tree algorithm or process as shown in flowchart of FIG. 6B. It computes a confidence ratio (ratio of rise in force magnitude from valley-peak to fall in force magnitude from peak-next valley) for each valley to peak to next valley (Vᵢ-Pᵢ-Vᵢ₊₁) combinations.

Flow chart representations for treatment stroke detection and rate estimation are presented in FIGS. 6A and 6B as explained above. Specifically, FIG. 6A is a flowchart showing a process 600 for identifying local maxima (Force Peak) and local minima (Force-Valley) from gradients of F_{RMS} data stream. In step 602, a F_{RMS} stream is imported from .csv file. In step 604, the F_{RMS} stream is sliced, after which low-pass filtering is performed on the sliced stream in step 606. In step 608, initialization is performed, setting each of the parameters as used in the subsequent steps in its initial stage. As used herein, tₛ is the treatment activity start time, tₛ+n is the treatment activity end time, F_{RMS(k)} is the current 3D resultant force, ΔF_{RMS} is defined as the difference between F_{RMS(k)} and F_{RMS(k-1)}, F_{Min} is the local minima of F_{RMS}, F_{Max} is the local maxima of F_{RMS}, k is the loop counter, n is the treatment activity length, i is the peak counter, and j is the valley counter.

After initialization in step 608, the process proceeds to step 610 to determine if k is less than n. If so, the process proceeds to step 614; otherwise, the process returns the arrays as shown in step 612. In step 615, the process determines if ΔF_{RMS} is less than 0. If so, the peaks are updated in step 616 as shown, after which the peak counter i is incremented in step 618, after which the loop counter k is incremented in step 626. Otherwise, the process proceeds to step 620, which determines if ΔF_{RMS} is greater than 0. If so, the valleys are updated in step 622 as shown, after which the valley counter j is incremented in step 624 and then the loop counter k is incremented in step 626. Otherwise, the process proceeds directly to increment the loop counter k in step 626. Following step 626, the process loops back to step 610 as explained above.

FIG. 6B is a flowchart showing a process 650 for performing decision tree-based algorithm to eliminate redundant force peaks per force-motion cycle due to hand vibrations or underlying irregularities in skin surface. This ensures each filtered peak as a stroke peak of that force-motion cycle and the stroke counts are summed up throughout the active time sequences to estimate the treatment rate of the session.

In the flowchart, Rise(i) is the current rise in force magnitude from valley to peak, Fall(i) is the current fall in force magnitude from peak to valley, R_{avg} is the average of rise array, F_{avg} is the average of fall array, C₍ᵢ₎ is the current confidence ratio (rise by fall), C_{Th(l)} is the confidence threshold lower limit, C_{Th(u)} is the confidence threshold upper limit, Gᵢ is the current gradient, Th is the rise/fall threshold, Pₘₐₓ is the maximum peak in the test array, i is the loop counter, j is the test array counter, k is the redundant peak array counter, n is the length of test array, S is the length of redundant peak array, T_Arr is the test array for temporary memory, and R_Arr is the redundant peaks array.

Specifically, while the process is running (while true) as determined in step 651, valley array is obtained in step 652 and peak array is obtained in step 653, both of which results from the process 600 explained above. These would be the input arguments to be read in steps 654 and 655, respectively. Subsequently, Fall(i) is determined in step 656, Rise(i) is determined in step 657, R_{avg} is determined in step 658, F_{avg} is determined in step 659, and C₍ᵢ₎ is determined in step 660. In step 661, the process determines if Rise(i) >(Th×R_{avg}) or Fall(i) >(Th×F_{avg}). If at least one is true, the process proceeds to step 662 to determine if C₍ᵢ₎ is between the values of C_{Th(i)} and C_{Th(u)}. If true, i is incremented in step 663 after which steps 654 and 655 are performed again. Otherwise, in step 664, C₍ᵢ₎ is analyzed such that if it is less than C_{Th(l)}, Gᵢ value is determined as −1 in step 665; otherwise, in step 666, C₍ᵢ₎ is analyzed such that if it is greater than C_{Th(u)}, Gᵢ value is determined as 1 in step 667. Afterwards, in step 668, if Gᵢ is determined to be greater than Gᵢ₋₁, Pₘₐₓ is set as shown in step 669, then step 670 is performed to determine Pₘₐₓ based on the population of T_Arr, and in step 671, S and R_Arr are determined, after which in step 672, T_Arr is reset. Subsequently, step 663 is performed.

If the decision is false in step 668, step 673 is performed to append T_Arr with the peak value of the loop counter i, then j is incremented, and G is updated as shown, after which step 663 is performed. If the decision is false in step 661, step 674 is performed to append R_Arr with the peak value of the loop counter i, then k is incremented, after which step 675 is performed to determine if i is greater than the length of PeakArr, which is the array to store the resultant of 3D force peak values, which is an input to the decision tree algorithm. If not, the process proceeds to perform step 663. Otherwise, step 676 to sort R_Arr and break/end the loop, after which in step 677, R_Arr is removed from PeakArr, and the filtered peaks are returned as the number of strokes in step 678.

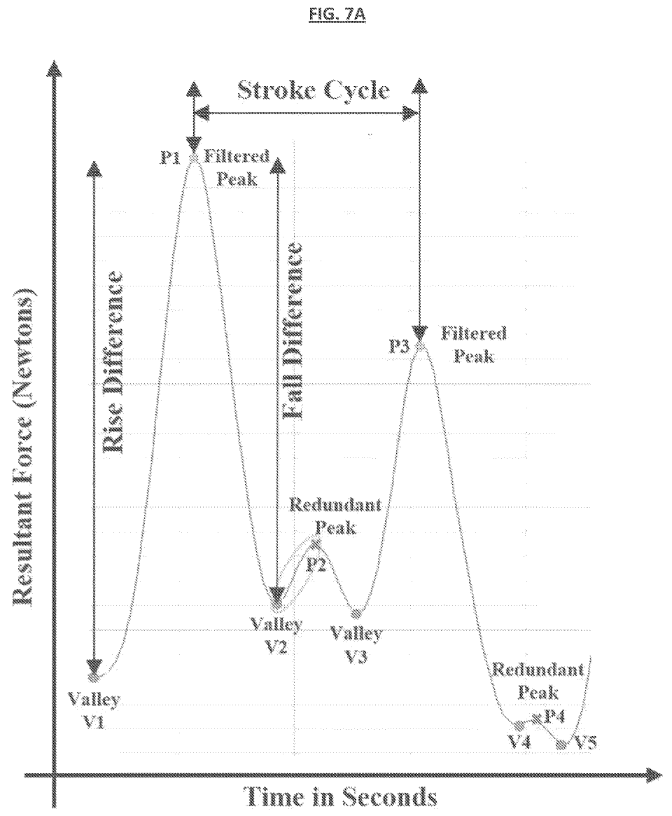

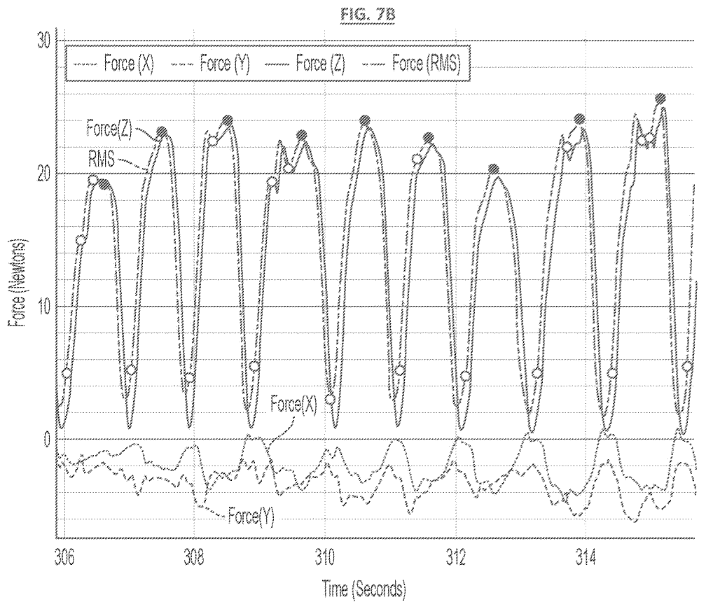

FIGS. 7A through 7C show force and motion waveforms as examples of multimodal graphical waveforms. FIG. 7A shows a sliced resultant force waveform, indicating peak-valley pairs and distinguishing primary stroke peaks and redundant peaks. FIG. 7B shows a dynamic waveform of time dependent 3D forces with FIG. 7C showing a dynamic waveform of corresponding 3D angular orientations depicting a force-motion progression.

The confidence ratios for each combination are further thresholded with a range of confidence thresholds (determined experimentally based on graphical observations of force waveform patterns) to discard redundant peaks, shown in FIG. 7A, thus conserving the primary peak force constituting every stroke cycle. The output of the algorithm yields the number of filtered peaks as number of strokes for the total contact time. A limitation of this technology in capturing the treatment motion-path traversed by the device with respect to the human body as an external reference is realized. Currently, the angular motion of the device is visualized by the change in yaw-pitch-roll data with respect to the stroke cycle. The stroke cycle is variable and depends on the volitional adaptations of motions of the user based on treatment. FIG. 7B represents the 3D force curve, and FIG. 7C represents the 3D angular orientation (Geo-Angle) curve, both with respect to time in the form of waveforms taken from a treatment window. Distinct repetitions of change in 3D angular orientations of the device are evident from Geo-Angle curve in FIG. 7C. Such repetitions form similar motion patterns performed by the therapist during a treatment.

The summation of strokes over a sequence of contact times for each force-motion portfolio is then calculated and divided by the total treatment time to yield the Treatment Stroke frequency which indicates the treatment rate. This information, along with the target force and the average treatment angle, is critical for determining the treatment type (STM stroke types applied) for personalized STM treatments.

### Experiments and Results

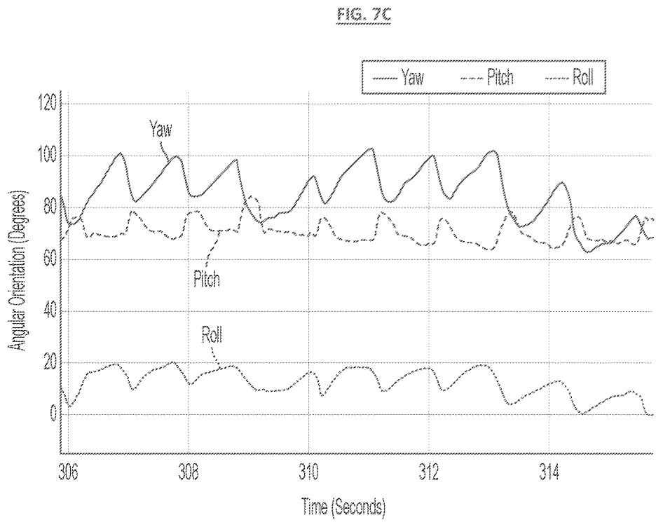

For experimental purposes, two different versions of dispersive handheld devices were built with maximum 200N and 400N force measurement capacities, out of which the former saturates within 160N-180N compressive force range, while the latter measures up to 325N-360N range as shown in FIG. 8. The device's estimated 3D rotation angles were validated by placing it on a manually operated pan-tilt calibration test rig. Several experiments have been performed to validate measured forces applied with different handgrips, especially, double handed grip and single-handed handhold. Moreover, measured steady forces were validated on an external force plate (PCE-PB-150N), of 0.5N measuring resolution, placed on the base of the test rig.

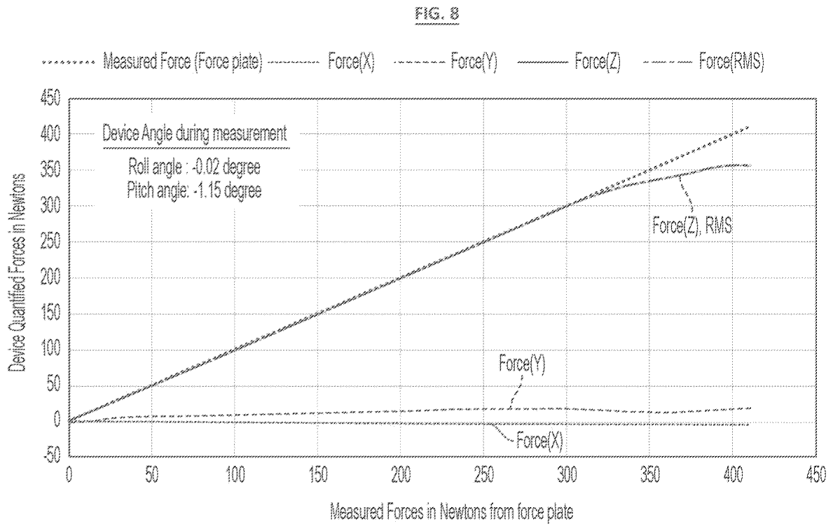

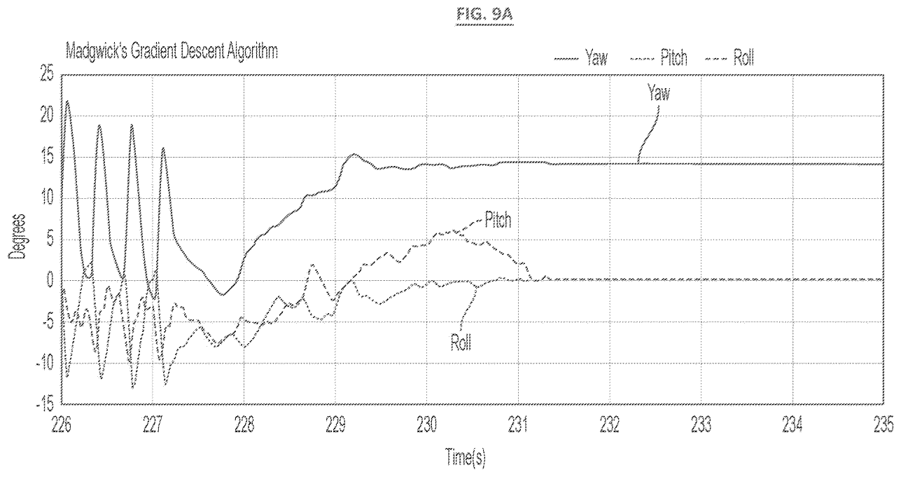

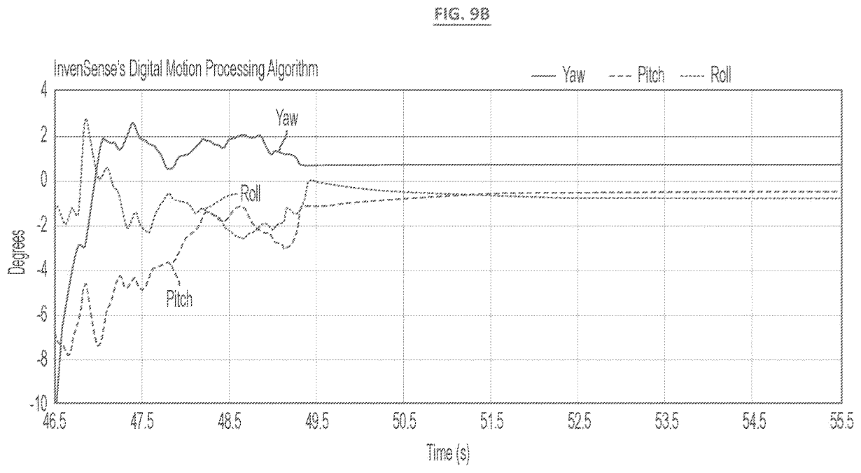

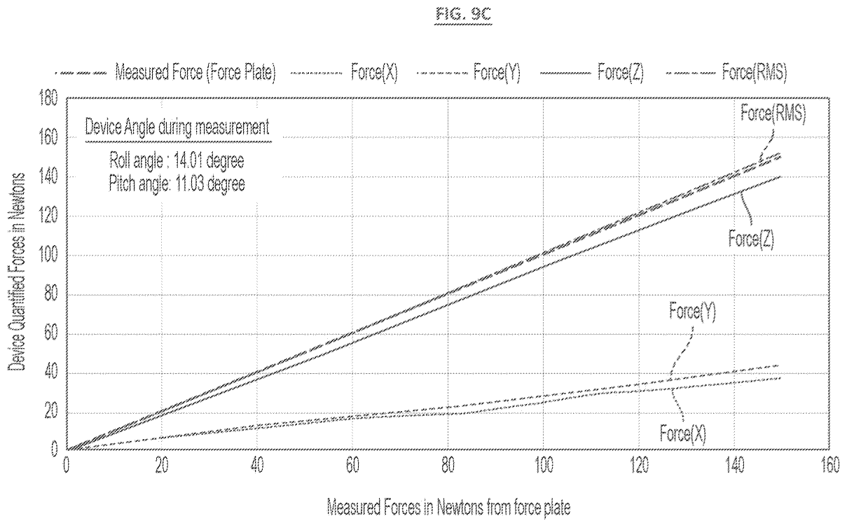

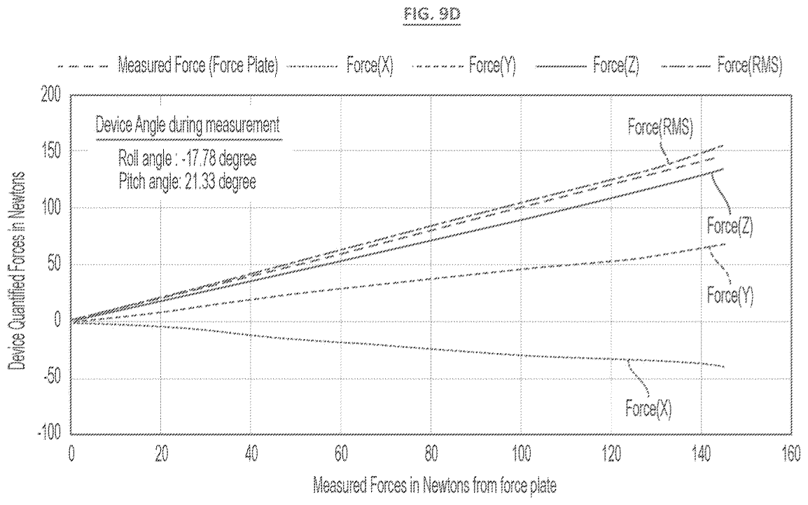

The load cells operate on 3.3V DC power, and the 16-bit analog to digital converter quantizes the measured voltages in approximately 0.02-0.03 mV range. This translates to device's compressive (along Z axis) force resolution to be −0.1N to 0.2N range based on the manufacturer's calibration matrix (A_{3×3}), while that of planar (along X & Y axes) forces account to be about ±0.05N to ±0.1N range. The static and dynamic responses of Euler angle rotations were further validated on the Orientation Viewer of MATLAB's Sensor Fusion Toolbox and compared with its built-in Kalman filter based AHRS algorithms. The response of the Gradient Descent based orientation estimation AHRS filter proved to be effective for steady motions within 0-5 Hz range with a ±2.15% error range. Repeated observations of sporadic nondeterministic dynamic motion gestures (with jerks and flickers ˜>5 Hz) produces a rotational drift more than +10% error in measured Yaw angles, which adds up over prolonged usage. Implementation of InvenSense's Digital Motion Processing (DMP) algorithm, comparison shown in FIGS. 9A and 9B, effectively compensates this drift error reducing the error range to +2%. Additional techniques for absolute pose estimation can be achieved by fusing 3D (North, East, Down) components from Magnetometer data into MARG filter or Extended Complimentary AHRS filter, after calibrating for hard and soft iron offsets, introduced due to environmental electromagnetic interferences. The linearity of device's computed forces with respect to the measured forces on external force plate is evident from the graphs shown in FIGS. 9C and 9D. However, these graphs also reveal an approximate linearity in force measurement error with rising force magnitude measured at both orientations of the device. The error escalation can be minimized by observing the force response of the device mounted on a Robotic Arm, at different orientations and tuning the calibration parameters for optimal performance.

FIGS. 9A through 9D show comparisons of orientation algorithms with respect to dynamic non-deterministic motions and graphical representations showing linearity in force measurements at different angular orientations. FIG. 9A shows Drift of Yaw orientation angles calculated by Gradient Descent Algorithm, which does not converge to zero at initial orientation position after suffering vibrational motion. FIG. 9B shows improvements in orientation convergence by measurements from Digital Motion Processing Algorithm when device returns to initial position irrespective of vibrational motions. FIG. 9C shows force validation at a right inclined orientation, where F_{RMS} is compared with the measured forces in dashed line. FIG. 9D shows force validation at left inclined orientation, where 3D forces are compared to the measured force in dashed line.

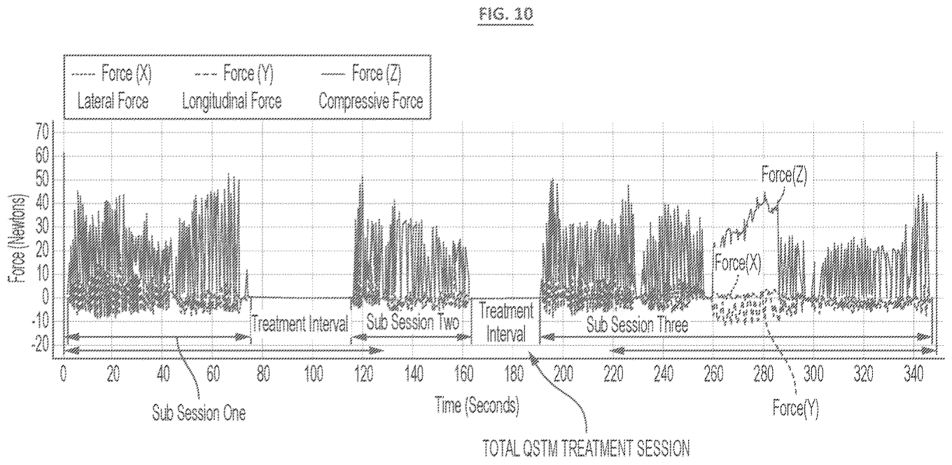

In prior implementations as known in the art, experiments with the handheld dispersive device were performed on both inanimate padded surfaces and in rodents. The Institutional Review Board of Indiana University under protocol number 1408895969 approved human subjects clinical trials on 6 Aug. 2021 for assessing the clinical impact of QSTM (in progress). FIG. 10 is a graphical depiction of 3D force waveforms representing six minutes of treatment session with handheld dispersive QSTM device. The figure also indicates the discreet treatment vsub-sessions (operational state), and treatment interval periods (pause state) in between sub-sessions during Treatment mode. Visual observations from the graphical 3D force-time waveforms as shown in FIG. 10 represents the operational state i.e., treatment sub-sessions, and the pause state (treatment interval in between sub-sessions) during a six-minute treatment session on a human subject. Different force-motion stroke patterns combining planar (longitudinal and lateral), and compressive forces, collectively constitutes the STM Dose-load regimen for consistent and variable frequencies over stipulated contact times, as administered by the clinician. The magnitude and frequency of these compressive and planar forces synchronously (in phase) or asynchronously (out of phase) applied on the skin directly impacts the underlying soft tissue properties. Hence, the average resultant force magnitude of every force-motion cycle and combinations of their 3D components defines a unit Dose-load per motion pattern. The force-motion patterns are of important clinical significance, as cells and tissues are highly sensitive to different external stimuli (compressive, tensile or shear stresses). The train of these consecutive similar paced force-motion stroke patterns within a sub-session are called treatment bursts in clinical terms. A series of treatment bursts constitute a treatment sub-session (operational state of device i.e., skin contact time between two pause states). The sequence of these treatment sub-sessions (operational state) and the interposing treatment interval (pause state) add up to the total treatment time called the treatment session.

The decision tree-based stroke count algorithm may be validated with manual counts per visual recordings to identify false positives (missed peak) and missed strokes over stipulated skin contact time intervals. The computation of stroke frequency and bursts occur at the pause state after every sub-session as a part of post processing. FIG. 11 is a graphical 3D force waveforms from “sub-session two” shown in FIG. 10 capturing linear (longitudinal cross fiber massage) and hybrid curvilinear (fanning motion) force-motion patterns which depicts discreet combination of planar and compressive force component waveforms. The treatment burst patterns (train of similar paced force-motion cycles) progressions illustrate the nature of the treatment sub-session. The RMS force peaks and valleys shows the accuracy of the decision tree-based stroke count algorithm, as every force peaks (green dot) corresponds to a single force-motion cycle. The red dots in FIG. 11 depict the F_{RMS} valley, while green dots are the peaks per stroke (force-motion cycle). Burst four in FIG. 11 represents a slow-paced curvilinear fanning motion. The red dots at the hill of second, third, fourth and successive strokes of burst four, indicates several redundant peak-valley pairs (due to hand vibration or soft tissue irregularity), which are successfully discarded to detect accurate stroke count and max peak per stroke in green dots. The stroke count algorithm proved to be 99% accurate when the device is used on inanimate objects, smooth tissue surfaces or rough tissue surface with slower rate as shown in FIG. 11. However, the accuracy level decreases to 90%, when the device is applied in varying orientations and directions over regions with uneven, curved, or non-uniform contours of human body (e.g., posterior thigh or calf muscle). Additional graphical observations also revealed that the accuracy of the stroke count algorithm varies with the rate of change of application i.e., the change in direction of stroke motions, change in contour of the treatment surface, or treatment pace. The average stroke frequency of every STM burst can be calculated to improve stroke count accuracy. Identification of a treatment burst can be computationally challenging for non-deterministic motions, as the user maneuvers and adapts to different force-motion portfolios at varying paces based on the instrumented palpation of the soft tissue region and treatment goal. FIG. 11 expands waveform of “sub-session two” shown in FIG. 10. It illustrates discreet combinations of different force-motion patterns with varying frequencies and directionalities of the planar force component progressions. Visual observations of bursts one and two from FIG. 11 indicates a cross fiber massage technique (i.e., linear back and forth motion parallel to the soft tissue fiber alignment) as the amplitude of the longitudinal force component along the y axis (in green) comprises a major part of the resultant force. While burst four show approximately consistent deflection from valley to peak along the y axis (longitudinal direction in green) for corresponding valley to valley forces along the z axis (compressive force component in blue), which illustrates that the device traversed a curvilinear path. This burst is called hybrid curvilinear motion as the frequency of the burst varies due to change of force amplitude as well as stroke length while performing a fanning motion with a single or double handed grip. Therefore, the dispersive handheld device enables identification of different treatment force-motion signatures, which can prove to be a clinical training tool or notation to reproduce and standardize dose-load regimens for replicable manual therapy.

To support the clinical usability of the developed dispersive QSTM device in quantifying treatment, a case study on a human subject with LBP was performed by an experienced manual therapist (>25 years of experience) under prior approval of the Institutional Review Board at Indiana University. The subject suffered low back pain (>1 year) from Lumbosacral grade-1 spondylolisthesis at L4-L5 segmental level with intersegmental disc degeneration evidenced by supporting radiographs. Four sessions were provided at 10 mins/session with 3-day intervals for 2 weeks using both the localized handheld QSTM device and the dispersive handheld QSTM device, previously elaborated in this paper, for treating the LBP condition, based on a standard IASTM protocol (GRASTON technique). The subject was not on any prescribed pain medications during the study. Functional and biological outcomes (trunk flexibility, soft tissue quality, and/or static pain pressure threshold “SPPT”) were measured before and after treatment for all sessions using standardized clinical procedures including the modified-Schober's test, MyotonPro, and handheld algometer, respectively. During SPPT testing, the subject was asked to indicate changes in pressure application from “comfortable to uncomfortable” by stating “now.” SPPT is inversely related to pain sensitivity. The average device to skin contact times were recorded to be 80.16% of total treatment time for combined use of both devices per session. The subject received a cold pack and instructions in gentle stretching exercises between sessions to reduce any potential soreness due to QSTM treatment.

The time taken by the device system from bootup to treatment ready state for the user to start STM application is approximately one minute, with an additional minute for adding post treatment remarks and bookmarking (2 minutes total). This time is reasonable with respect to clinical feasibility and information gained by using the dispersive QSTM device system. Documented QSTM treatment charts demonstrated force-motion patterns (linear types-Strumming and Scanning, Curvilinear types- Fanning and Sweeping) observed for a variety of treatment bursts of different stroke time-lengths and paces constituting a treatment session. The treatment force charts revealed initial pace building strokes during scanning the tissue followed by consistent force delivery for myofascial release. Average device to skin contact times were attributed to 47.22% for the localized device and 33.10% for dispersive device. Comparatively, the average STM dose regimen (average of resultant force peaks) was 2.4 times (137.5%) higher for dispersive device as compared to the localized one, whereas the force motion for the dispersive device were 41% slower with longer stroke lengths and a 20.6% steeper inclination to skin surface as compared to the localized device. Intra-session treatment report comparison showed 135% higher targeted force delivery on the last session as compared to the first. Improvements in soft tissue characteristics from first to last session were realized from the MyotonPro (9.9% less tissue stiffness, 3.4% less creep, 5.4% increased relaxation). The SPPT increased significantly across sessions (from first to last) representing a 73.58% increase in pressure tolerance i.e., lowered pain sensitivity at the most painful site, after the last session. Eventually, steady improvements on self-reported pain levels reached an average 0/10, and 2/10 worse pain level after the fourth treatment, down from an average 7/10 and 9/10 worst pain levels before first treatment session. The overall positive results and gradual pain level improvements documented in the case study establishes the clinical feasibility of QSTM medical device system for research and clinical use for reproducible manually therapy. However, clinical trials are needed to determine the fidelity and efficacy of this novel technology, and study dose-load response in a variety of NMSK treatment and interventions.

According to some examples, there is an offset voltage drift, triggered during repeated usage due to the load cell's loading-unloading characteristics. This ensures a force baseline (zero force at no-load) shift, which marks the necessity for self-calibration of the device during its pause state of treatment mode. For recalibration purposes, the device may rest in its cradle at its predefined orientation. Therefore, the firmware monitors the quaternion orientations during self-calibration for a no-motion time interval. If the mean orientation during this interval is near the initial calibration orientation, then the device undergoes automatic recalibration, and the calibration parameters are updated in the firmware memory. This process assists in baseline restoration for force measurements and compensates for any drift in the force offsets, preserving device sensitivity.

As such, in view of the above, some of the benefits offered by the presently disclosed system may include quantification of manual therapy using objective treatment parameters as a key to precision rehabilitation. The system according to some examples may offer both targeted STM dose-load delivery with software guided feedback as well as adaptable maneuverability by the practitioner, required for individualized care of NMSK conditions. The validation results show accurate quantitated force measurements and angular orientation estimation of the device with minimal error, post proper calibration. This quantifiable IASTM medical device system is practical for clinical use without significantly increasing the treatment time compared to hands-alone manual therapy. In some examples, the fidelity and precision of the device may enable accurate detection of stroke frequencies up to 5 Hz. In some examples, the force measurement accuracies work best within force measurement range of from 0.2 N to 325 N, inclusively, hence this medical device would be suited to quantify STM treatments for a varied spectrum of patients with high to low pain tolerances. Therefore, usability of the system is demonstrated, and positive outcomes are observed in an individual with low back pain, which may be evidenced, for example, by reduced self-reported pain levels in conjunction with elevated magnitude of dose-loads tolerated by the human subject at the last treatment session as compared to the first.

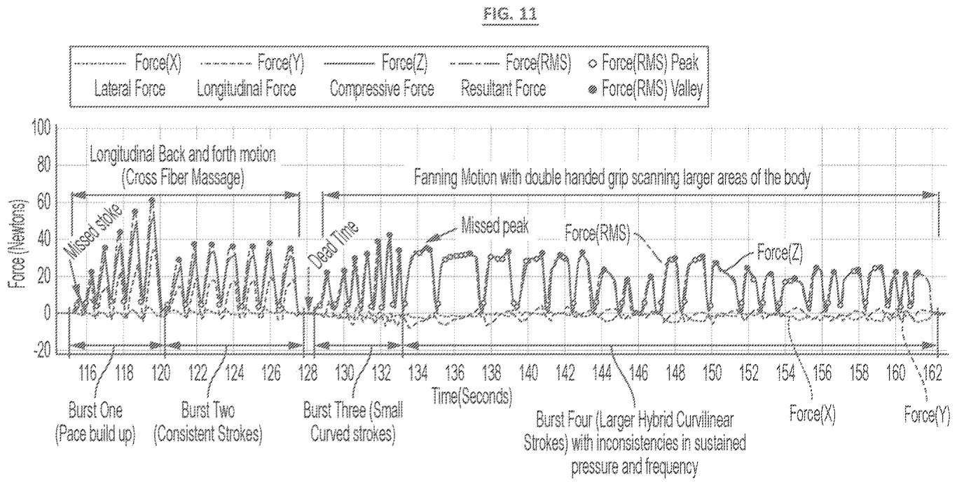

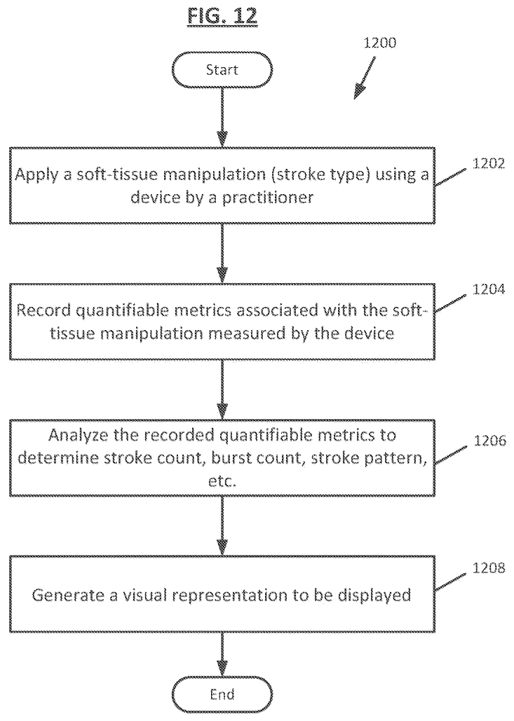

FIG. 12 shows a process 1200 which may be implemented according to embodiments disclosed herein. In step 1202 of the process, a soft-tissue manipulation i.e. one or more burst(s) of identical force-motion progression or sequence (STM Stroke type) at an particular pace or frequency is applied to a patient using any QSTM device 4 by a practitioner. The soft-tissue manipulation may be any one or more of the STM stroke types as further explained herein. The device may be any of the localized or dispersive force-motion applicators of the handheld QSTM device 4 as explained herein. In step 1204, the quantifiable metrics that are associated with the soft-tissue manipulation are measured by one or more sensor member 6 associated with or installed on the handheld device are transmitted to the display unit 8. The transmitted data are tracked in real-time by the computing device coupled to the display unit 8 in the form of graphical Force and motion waveforms. These measured quantities of force and motion are recorded and stored in the memory of the computing device of the display unit by the software (for e.g. Q-Ware) running on the display unit.

In step 1206, the recorded quantifiable metrics are analyzed, for example as a part of post-processing, to determine one or more factors including, but not limited to, any one or more of: stroke count (as explained herein in view of FIGS. 6B and 11), burst count (as explained herein in view of FIG. 11), and/or stroke pattern (as explained herein in view of FIG. 10 and FIG. 11). The analysis may be performed using any suitable method or algorithm as explained herein. The factors that are determined as a result of the analysis may be stored or saved in the same device or component that records the metrics as explained in step 1204, for example.

In step 1208, a visual representation of the force-motion waveforms in response to the soft tissue manipulation(s) or identical STM stroke types applied to the soft tissue as a part of treatment are generated along with recorded quantifiable metrics (for e.g. treatment report) on a Data Analysis Interface (DAI) window of the software (for e.g. Q-Ware) running on the computing device associated to display unit 8 is generated, or more specifically, data which can be displayed on a display such as the visual display 8. The generated graphical representation of force-motion waveforms may be observed by the practitioner to further compare, visually analyze, and/or review identical STM stroke patterns after each procedure or completion of a treatment session. The visual graphical representation may include information including, but not limited to, the factor(s) determined in step 1206. In some examples, the stroke pattern may be determined or identified by analyzing the waveforms generated as disclosed herein, and the stroke pattern may be analyzed to recognize the identical signature(s) of STM stroke type(s) as performed by each user or practitioner, for example by comparing the composition of angular orientation of force application, such that each stroke (identical force-motion pattern) can be mathematically labeled with a cost function for ease of statistical comparison between the two similar STM stroke types.

In some examples, step 1204 may be performed simultaneously or near-simultaneously (that is, in real-time or close to real-time, such as within 1 second, 0.5 second, 0.1 second, or any other suitable range of time therebetween) as step 1202 such that data is recorded as the user or practitioner is performing the soft-tissue manipulation.

In such examples, audio signals such as audio feedbacks may be provided by the device that is used for the soft-tissue manipulation when, during the application, the user or practitioner is applying a force that surpasses or exceeds a maximum determined threshold of force for the patient or a predetermined maximum threshold of force, so as to provide instantaneous or near-instantaneous warning or alert for the user or practitioner to reduce the force magnitude applied so as to prevent causing injury to the patient or maintain targeted pressure consistency. In another embodiment, a “target force trendline” may be set to a Force magnitude threshold on the real-time visual display, such that the practitioner watches the display unit repeatedly to maintain the force application magnitude to the set target force for targeted pressure consistency.

If two of the QSTM devices work in tandem with one another (the first device is used first, then the second device is used thereafter, e.g. in sequential repetition by the same user/practitioner to perform a multiple-device treatment session covering regional areas of the body) or if a single device is used by two users (the users/practitioners take turn using the same device), the system may be able to recognize or determine that two different users or practitioners are performing the soft-tissue manipulations based on the aforementioned analysis as explained herein, without having the users or practitioners provide such information. Such recognition may be possible using methods as disclosed herein to identify the stroke pattern that is unique to each of the manipulations, for example by determining a first stroke pattern for the first soft-tissue manipulation and a second stroke pattern of the second soft-tissue manipulation. In some examples, the system may handle more than two users and/or more than two devices (QSTM devices and/or force-motion applicators), such as three or more users using two devices, three or more users using one device, or three or more users each using a separate device, such that there may be three or more sets of data to analyze and display, for example. In some examples, the user or practitioner may be prompted to document treatment remarks about the treatment sub-sessions on the remote device via an interactive visual display (e.g., visual display 8) before saving the treatment report of the performed treatment session. In some examples, the QSTM device(s) may also facilitate the user or practitioner to apply the soft-tissue manipulation stroke type(s) by the same QSTM device performing a single-device treatment session.

For example, as explained with respect to FIG. 5A, the Treatment mode of the system may have four (4) states: calibration, ready, operational, and pause. The system may perform self-calibration using the calibration state (and based on force/angle data as explained herein) such that the device re-calibrates itself when the user or practitioner take turns with using the device to perform the soft-tissue manipulations. That is, the automatic calibration takes place when the device detects that the first soft-tissue manipulation (performed by the first user, for example) is completed and the device is in pause state (not in motion or exerting any forces), such that the automatic calibration can take place before the second soft-tissue manipulation (performed by the second user, for example) can begin or initiate. However, the calibration would not take place during the manipulations in order to preserve accuracy of the measurement that is taken during the manipulations.

As such, using the methods or algorithms as explained herein, the system may also perform automatic continuous real-time (or near real-time) weight correction to achieve a force measurement range of from 0.2 N to 200 N, inclusively, based on the force/angle data as measured. In some examples, the range may be from 0.2 N to 250 N, from 0.2 N to 275 N, from 0.2 N to 300 N, from 0.2 N to 325 N, inclusively, or any other suitable range or value therebetween.

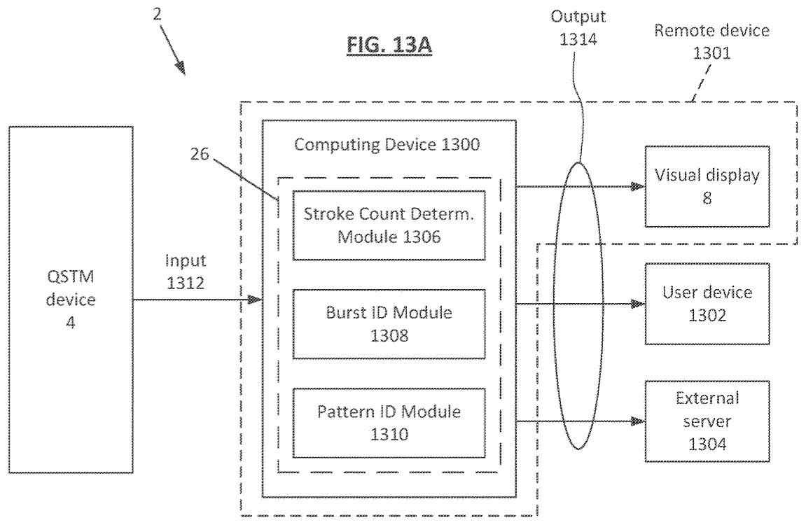

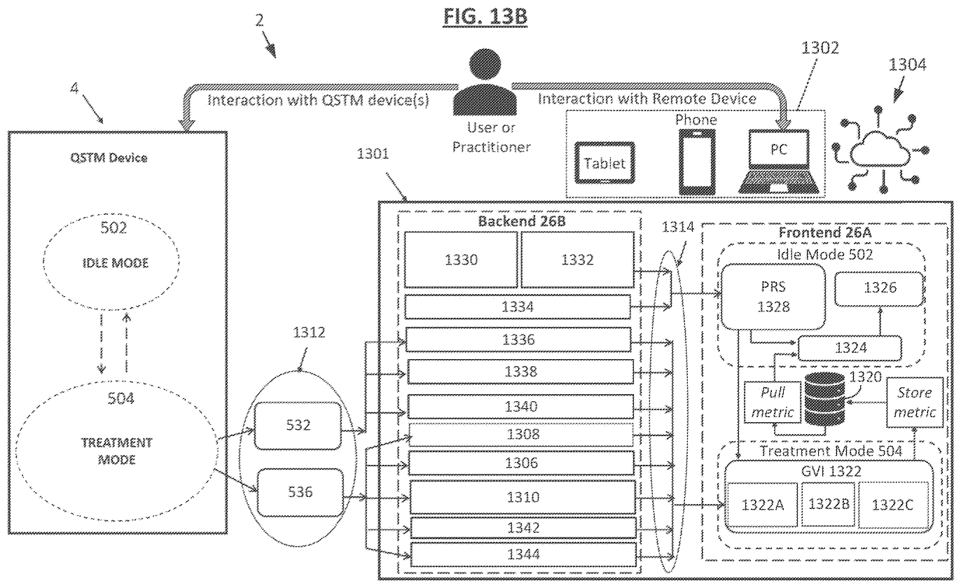

FIG. 13A shows a high-level block diagram of the system 2 according to some examples which includes the QSTM device 4 as disclosed herein, and a remote device 1301. The user or practitioner may interact with both the QSTM device 4 and the remote device 1301 separately. The remote device 1301 includes a computing device 1300, which is operatively coupled with the visual display 8 of the remote device 1301. Also, in some examples, an additional user device(s) 1302 such as a smartphone and an external server 1304 for remote access of data may be operatively coupled with the computing device 1300 to receive data. The computing device 1300 includes a plurality of software components 26. The components 26 may be implemented as separate modules as shown or as a single module or processing device which is capable of performing all the functionality as explained herein. The components 26, according to some examples, include a stroke count determination module 1306 which performs the process 600 shown in FIGS. 6A and 6B. The components 26 also include a treatment burst count determination or treatment burst identification module 1308 which determines the treatment bursts of different force-motion patterns associated to different STM stroke types as shown in view of FIG. 11. In some examples, the burst counts of a progression of soft-tissue manipulation strokes are determined from the multimodal graphical waveforms and corresponding stroke counts applied in different stroke frequencies as a part of the treatment reports of the treatment session. In some examples, each of the burst counts is determined based on one or more of: (a) computed 3D force measurements including compressive, shear lateral, and shear longitudinal force measurements, (b) angular motion measurements including yaw, pitch, and roll measurements, (c) data of the 3D motions including 3D accelerometer values, 3D gyroscope values, or 3D magnetometer values, or (d) start and stop timestamps of the treatment session as provided using any suitable means as disclosed herein. In some examples, each of the burst counts may be determined based on thresholds of a decision tree comprising one or more of: computed 3D force measurements including compressive, shear lateral, and shear longitudinal force measurements, or start and stop timestamps associated with the burst counts, as explained herein. The components 26 also include a pattern identification module 1310 which determines or classifies identical STM stroke types from associated treatment force-motion waveform patterns using a process 1400 as shown in FIG. 14, as further explained herein. The computing device 1300 receives input data 1312 from the device 4 and processes them in the computing device and generates output data 1314 to be provided to the visual display 8 (for e.g. Q-Ware running on PC), user device 1302 (for e.g. Q-Ware running on smart phone or tablet), and/or external server 1304 (for e.g. Q-Ware addon database on a cloud storage).

In some examples, the input data 1312 includes one or more of the following: 3D force values, angular motion values (yaw, pitch, and roll), 3D accelerometer values, 3D gyroscope values, 3D magnetometer values, and/or the start/stop time of the device 4. The output data 1314 includes treatment stroke count data, treatment burst (consecutive force-motion waveforms with similar characteristics) count output data, and number of distinct STM stroke types determined from waveform patterns of the treatment session output data, as explained herein. According to some examples, the treatment burst count output data may be generated using any suitable supervised or unsupervised dataset training method such as a decision tree-based algorithm and/or a feedforward neural network, among others.

FIG. 13B shows a more detailed block diagram of the software architecture of system 2 according to some examples which includes at least one of the QSTM device 4 as disclosed herein, which applies a massage therapy on a soft tissue of a patient. The massage therapy includes soft-tissue manipulation stroke type(s) applied by a single practitioner during a treatment session. According to some examples, the QSTM device 4 (which may be a handheld mechatronic force-motion applicator as explained herein) may include RGB LED light(s) (e.g. the LED 27 of FIG. 4) to visualize the operation modes (e.g., the idle mode 502 and the treatment mode 504) and the working states of the QSTM device 4, as well as a control button (e.g. the control button 25 of FIG. 4) to be operated by the practitioner to change operation modes or working states of the QSTM device 4 during the treatment session. The QSTM device 4 also includes at least one treatment edge (e.g. the treatment edge 23 of FIG. 4) mechanically coupled with the at least one force or motion sensing unit such as the sensor member 6. The sensor member 6 measures quantifiable metrics including 3D motions and magnitude of compressive and shear forces applied during directional hand movements of the QSTM device 4 for the soft-tissue manipulation by performing the soft-tissue manipulation stroke type(s) using the treatment edge.

The QSTM device 4 may also include a computing device which include a processing unit (e.g., the processing unit 15 of FIG. 3) which is coupled with the sensor member 6 to RMS force data from the measured compressive and shear forces, and angular orientation data from the 3D motions including one or also includes a memory unit (e.g., the memory unit 29 of FIG. 4) which stores sensor calibration information and records data of the compressive and shear forces as measured by the sensor member 6 as well as treatment timestamps, in order to quantify the soft-tissue manipulation stroke type(s) performed on the patient. The QSTM device 4 may also include a receiver/transmitter (e.g., the receiver/transmitter 10 of FIG. 3) which may be coupled with the computing device to transmit and/or receive information data, as suitable. For example, the receiver/transmitter may be activated during the treatment 504 to send the input 1312 for the remote device 1301 as explained further herein.

The remote device 1301 may be a smart tablet, a smartphone, a personal computer, and/or any other suitable device coupled with an online server, for example. Furthermore, the additional user device(s) 1302 may include one or more additional smart tablet, smartphone, personal computer, or any other suitable device. The input 1312 may include the QSTM message string (as per step 532 explained above) and device pause message string (as per step 536 explained above). The remote device 1301 receives the data of the compressive and shear forces, the RMS force and angular orientation data, and the treatment timestamps transmitted from the QSTM device 4, and in response generates graphical data in the form of multimodal graphical waveforms for a visual, numeric, or statistical comparison of the quantifiable metrics associated with the soft-tissue manipulation stroke type(s) performed by the practitioner during the treatment session. The remote device 1301 also executes a software program for QSTM-based electronic treatment record (such as Q-Ware) to generate treatment reports and to document the treatment sessions. These functionalities of the remote device 1301 are facilitated using the software components 26 of the remote device 1301.

The software components 26 of the remote device 1301 may include a visual display frontend 26A associated with the visual display 8, and the frontend 26A includes a local database 1320 physically located on internal volatile primary or external secondary permanent memory of the remote computing device to store patient specific treatment reports, documentations and comparisons which may be any suitable memory unit including but not limited to static random access memory (SRAM), dynamic random access memory (DRAM), non-volatile flash memory, hard disk drive, solid state drive, etc. The local database or folder (directory) structure or memory address 1320 may save and record the generated treatment reports and the quantifiable metrics of the massage therapy in one or more treatment sessions performed by one or more practitioner on corresponding patients for treatment data organization and maintenance. That is, the local database or folder (directory) structure or memory address 1320 may store treatment metrics such that, in treatment mode 504, treatment metrics can be provided to be stored in the local database memory address space 1320, and in idle mode 502, the stored treatment metrics may be pulled or retrieved from the local database memory address space 1320. The frontend 26A may include GVI 1322 as shown in FIG. 2C which may receive the output 1314 and provide one or more of: live 3D geo-angle graph 1322A, live 3D force graph 1322B, and/or treatment parameter table 1322C, among any other suitable data or information as disclosed elsewhere herein. Furthermore, the frontend 26A includes a treatment retrieval manager 1324 to pull or retrieve the treatment metrics from the local database memory address space 1320 to provide to a data assessment interface 1326, as suitable. The frontend 26A may also include Patient-Treatment Record System (PRS) 1328 for patient entry or selection to start the operation of the Software (for e.g. Q-Ware) for treatment mode. The PRS 1328 also administers treatment data retrieval form recorded history of treatments using the treatment retrieval manager 1324 to compare and analyze multimodal signature waveform patterns of identical or individual STM Stroke types performed by inter or intra-therapist(s) on the Data Analysis Interface (DAI) 1326 of the associated software program (for e.g. Q-Ware). The output of the patient selection module, treatment retrieval module or Treatment waveforms assessment module from the backend processes of the software, as a part of user actions, are inputs to the associated processes of the PRS which is operably and logically coupled to the GVI 1322 of the STM quantification software program (for e.g. Q-Ware).

The software components 26 of the remote device 1301 may also include a series of backend processes 26B which includes the stroke count determination module 1306 shown in FIGS. 6A and 6B, the burst identification module 1308, and the pattern identification module 1310. There may be additional modules, as suitable, in the backend processes 26B to assist in generating suitable output 1314 for soft tissue treatment quantification, monitoring and characterization. For example, the backend 26B may include a patient entry or patient search module 1330 to enter or search information pertaining to the patient receiving the treatment. A treatment retrieval module 1332 may be operable to retrieve information regarding the treatment that the patient has previously received, or to pull up past patient record for treatment comparison purposes, for example. A treatment waveforms assessment module 1334 may be provided to assess the treatment waveforms to visually observe and determine information regarding the waveforms as disclosed herein. A live sensor data logging module 1336 may be provided to log the sensor data in or near real-time to provide real-time data log or live animated graphical visualization of the data stream of QSTM message string 532 for visual feedback. A live data storage module 1338 may be provided to store the data provided in or near real-time so as to be accessed easily by the frontend 26A. A data filtering module 1340 may be provided to filter the sensor data that is provided or retrieved. A device self-calibration module 1342 may be provided to facilitate the self-calibration of the device as disclosed herein. For example, when the QSTM device 4 detects that a soft-tissue manipulation is completed and the QSTM device is in a rest state or placed on its cradle, the QSTM device 4 may perform an automatic self-calibration on the series of sensors member 6 before the QSTM device 4 is used for a second time (for another soft-tissue manipulation) in the same treatment session. A treatment report generation module 1344 may be provided to generate a treatment report to be provided to the frontend 26A to be displayed or recorded, for example. The output 1314 to be provided to the frontend 26A may include any one or more of the outputs from these modules. Furthermore, any other suitable operation associated with the remote device 1301 or the QSTM device 4 may be facilitated using any other or additional module(s) as appropriate.

FIG. 14 shows the steps of a process 1400 used by the pattern identification module 1310 according to examples disclosed herein. In step 1402, the module or the computing device 1300 receives input data from the device 4 as previously explained. In step 1404, based on the input data, the module or computing device performs dimensionality reduction of the input data points, for example data slicing, to obtain maxima (peak points) and minima (nadir points) of the data points, as well as the timestamps of such maxima and minima. In step 1406, the module or computing device performs feature extraction on the obtained maxima and minima of the data points to obtain characteristic features. In some examples, the characteristic features (also referred to as graph features or attributes) include a amplitude gradient of peak-nadir pairs, confidence ratios of amplitude gradients and/or phase differences of peak-to-peak time correlation of different multimodal force and motion channels, such as the force and motion in the x, y, and z directions. The peak-to-peak time correlation is defined as a possible correlation (or a lack thereof) between peaks (maxima) of different data sets, including data points of compressive force (z direction), shear lateral force (x direction), shear longitudinal force (y direction), and root-mean-square thereof, a.k.a. the resultant force.

In step 1408, the module or computing device performs pattern identification using machine learning (such as feedforward or deep recurrent neural network, reinforcement learning, support vector machines, wavelet transforms, template matching or any other suitable type of machine learning approaches known in the art to classify STM stroke type into signature identical force-motion waveform patterns, for example) based on the characteristic features obtained in step 1406, in order to determine a current treatment force-motion waveform pattern as classified STM Stroke type.

In some examples, the process may proceed from step 1408 to step 1412 directly, or step 1410 may take place in between. In step 1410, the module or computing device or remote device 1301 compares the current treatment force-motion pattern with a previously determined (or stored) treatment force-motion pattern to determine a percentage match of the two-treatment force-motion waveform patterns. Based on the percentage match, the module or computing device may further identify the points in the current treatment force-motion pattern which may be similar to or different from the previous treatment force-motion pattern, to be reviewed by the user.

In step 1412, the module or computing device outputs the current treatment force-motion pattern to a display unit coupled with a remote device 1301. Optionally, if step 1410 has been performed, the percentage match and/or the overlapping visual comparison of the current and previously determined treatment force-motion patterns may be displayed on the display device to be reviewed by the user. In some examples, the current treatment report, types of STM strokes used in the session determined from associated force-motion pattern, and the degree of the percentage match of the identical force-motion waveform pattern with respect to similarly known previously determined patterns may also be sent to the user's own device and/or a remote server to be accessed by other users.

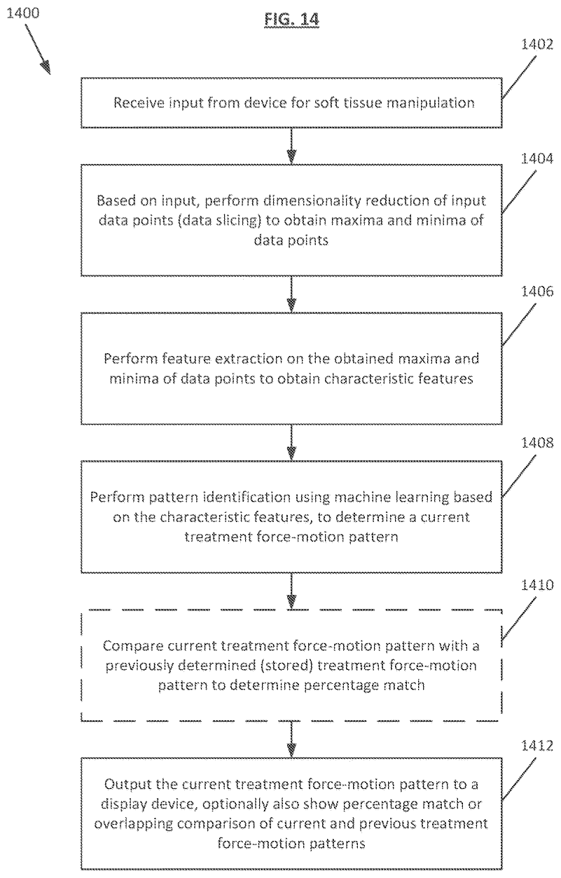

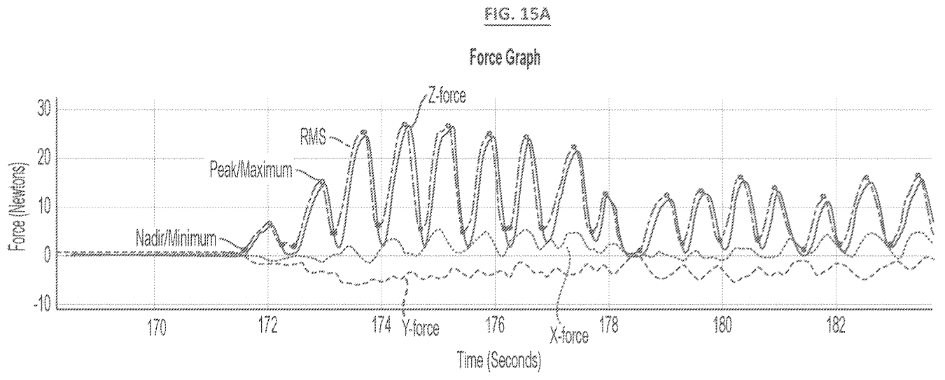

FIG. 15 shows an example of the force versus time graph and angular orientation versus time graph with the maxima/peaks and minima/nadirs of each of different data channels sensed by the QSTM device depicting RMS, yaw, pitch, and roll, as explained in step 1404 of FIG. 14 as explained above. In the example shown, only the maxima and minima of the RMS are determined for use in the pattern identification process, but in some examples, the maxima and minima of the x-force, y-force, and z-force may be taken into consideration as well.

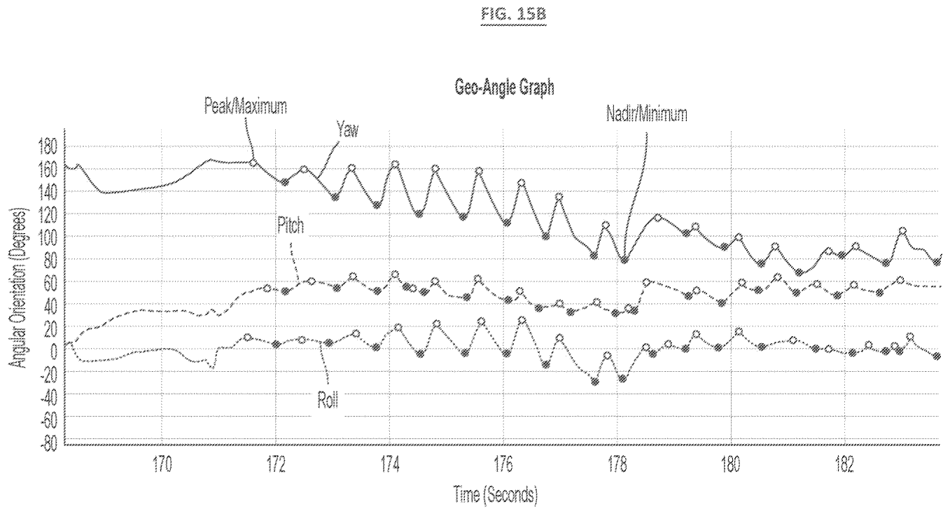

FIG. 16A shows an example of the QSTM device 4 that is localized, i.e. a localized force-motion applicator device with a half disc shaped tooltip that is configured to apply force at a given area and take measurements locally at smaller area body regions. FIG. 16B shows an example of the device 4 that is dispersive, i.e. a dispersive force-motion applicator device with a wide treatment blade that is configured to apply pressure and take measurements over a wider body area than the tip of the localized force-motion applicator device can achieve. FIG. 16B also shows a treatment on a low back pain subject/patient having grade-I spondylolisthesis marked with location that is determined as the most painful spot for the patient, as well as static pain pressure threshold measurement spots from pain algometry. For example, the biological outcomes (trunk flexibility, soft tissue quality, and static pressure pain threshold (SPPT) may be measured before and after treatment for all sessions using standardized clinical procedures using modified-Schober's test, MyotonPro, and handheld algometer, respectively.

As such, the user may implement the aforementioned two ergonomically designed, portable, handheld smart medical devices (a localized STM applicator of FIG. 16A with a half-disc shaped contoured steel tip and a dispersive STM applicator of FIG. 16B with broader convex steel treatment edge for tissue palpation/scanning and treatment. The device 4 captures real-time 3D (compressive, transverse longitudinal, and transverse latitudinal) forces and 6D (that is, 3-axis linear and 3-axis rotational) motions of STM practice as clinically adapted by the user during treatment, which are transmitted to a computer software (e.g., Q-Ware) for real-time graphical visualization for feedback. The software may offer treatment data recording for comparison and guided reproducibility, performance assessment of user to curb practice inconsistencies and identifies the STM Dose regimen applied to the patient for a specific biological outcome. To support the feasibility and efficacy of QSTM technology, a controlled case study on a human subject with Low Back Pain (Lumbosacral grade-1 spondylolisthesis at L4-L5 segmental level with intersegmental disc degeneration from supporting radiographs) may performed by an expert manual therapist for a predetermined number of sessions (e.g., 4 sessions) for a predetermined duration (e.g., 10 mins/session) with a predetermined number of days in between (e.g., 3-day intervals for 2 weeks). The subject may not be under any prescribed pain medication during the study. Such case study was performed with prior approval of the Institutional Review Board at Indiana University. The biological outcomes (trunk flexibility, soft tissue quality, & static pain pressure threshold SPPT) were measured before and after treatment for all sessions using standardized clinical procedures using modified-Schober's test, MyotonPro, & handheld algometer, respectively. The average device to skin contact time were recorded to be 80.16% of total treatment time for combined treatment of both devices per session, while treatment procedures were based on standard IASTM protocol (GRASTON technique).

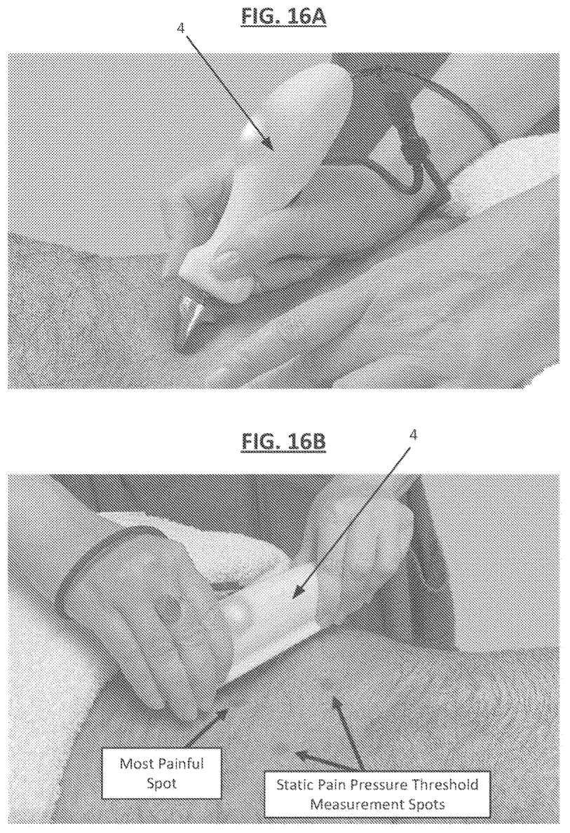

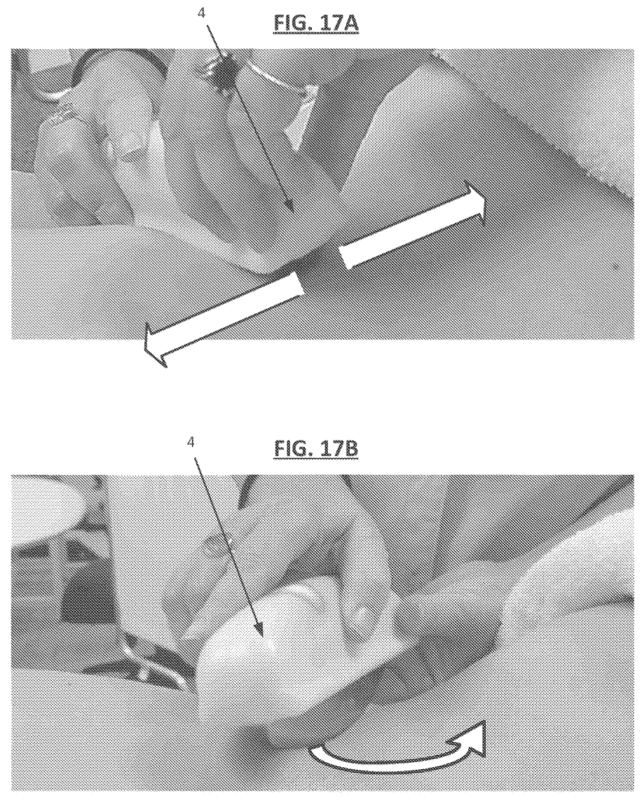

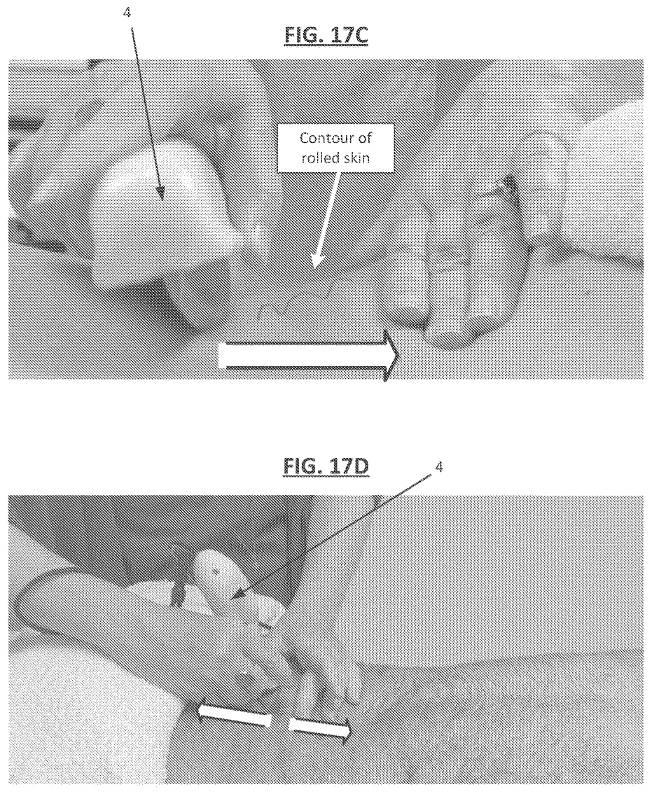

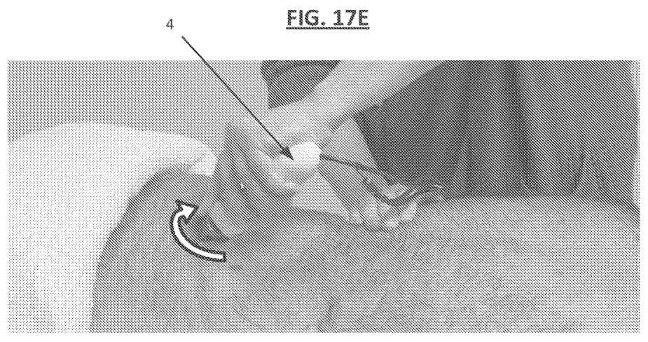

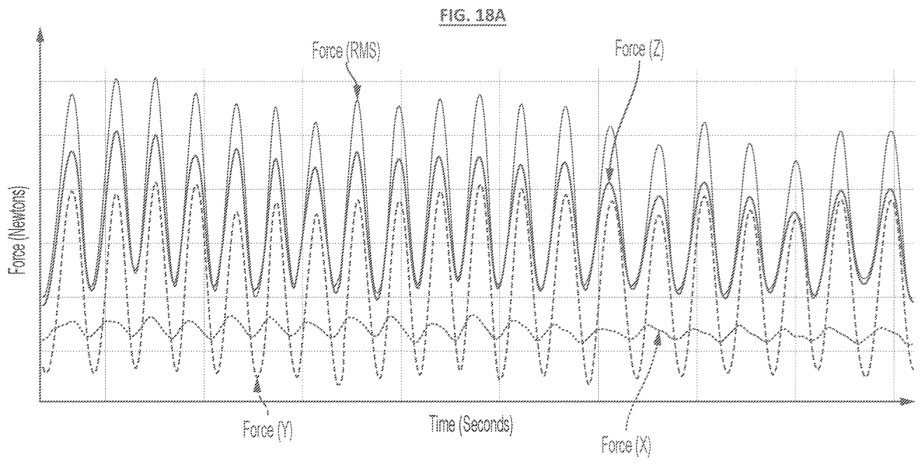

As the clinical findings demonstrated, the system advantageously improves consistent targeted force delivery with software guided visual feedback, where different force-motion patterns as disclosed further below with respect to FIGS. 17A through 17E, observed for a variety of bursts (train of similar stroke patterns) of different stroke lengths and paces constituted a treatment session. Contact times of 47.22% for the localized applicators as compared to 33.10% for the dispersive applicators were attributed for the treatment sessions approximately. Comparatively, the average STM dose regimen (average of resultant force peaks) was 2.4% (137.5%) higher for dispersive device as compared to the localized one, whereas the force motions for the dispersive device were 41% slower with longer stroke lengths and 20.6% steeper inclination to skin surface as compared to the localized device. The improvements in soft tissue characteristics from first to last session (9.9% less stiffness, 3.4% less creep, 5.4% increased relaxation) were realized from MyotonPro, while the subject's self-reported pain level on a 0-10 Likert scale (0=no pain, 10=worst pain) were documented as well. The SPPT increased vehemently across sessions (from first to last) representing 73.58% pressure tolerance with low pain sensitivity at the most painful site after the last session. Eventually, steady improvements on self-reported pain levels reached an average=0/10 with 2/10 worse pain after the fourth treatment, from average 7/10 upon longer (e.g., greater than 15 minutes) sitting or standing position with 9/10 being the worst pain, before first treatment session.

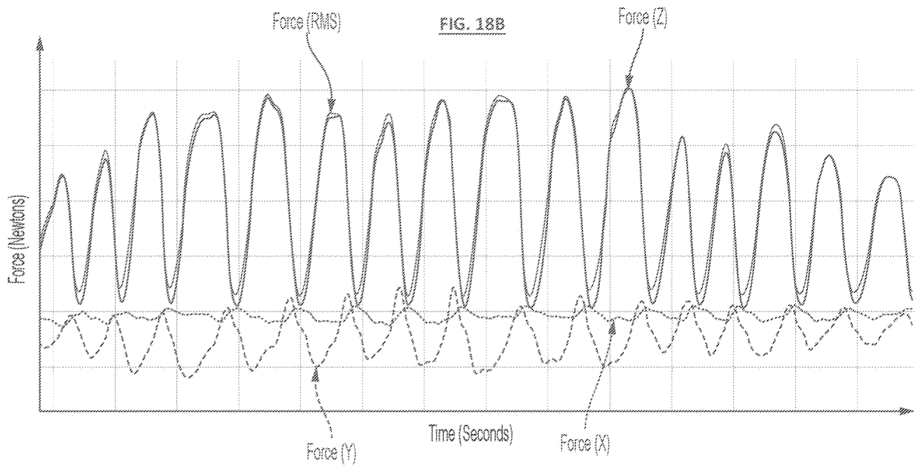

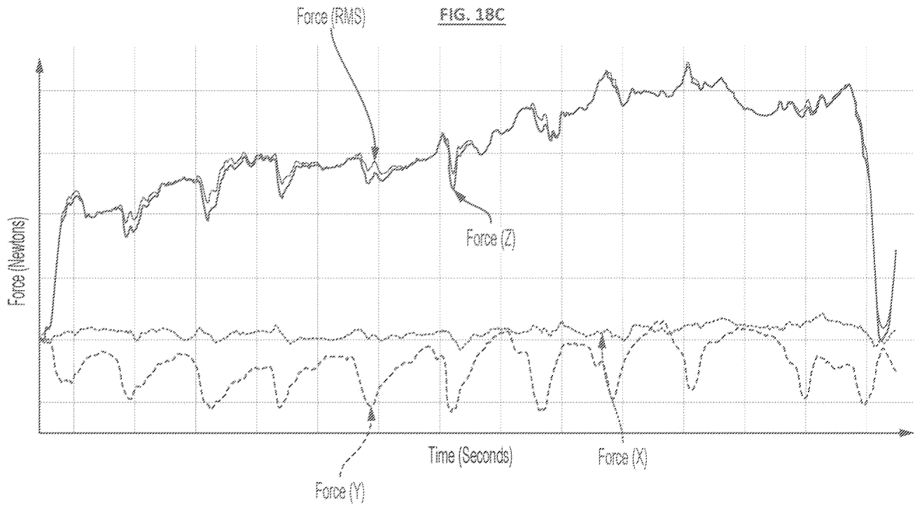

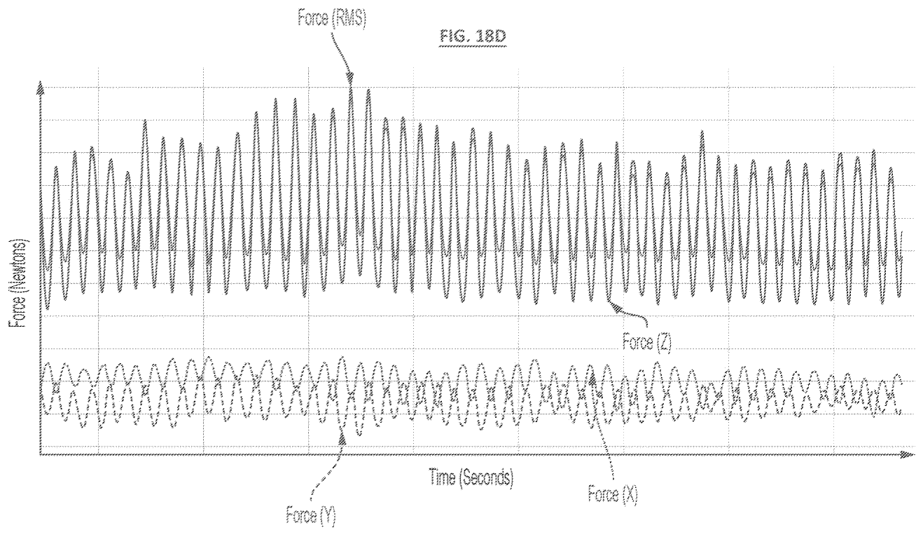

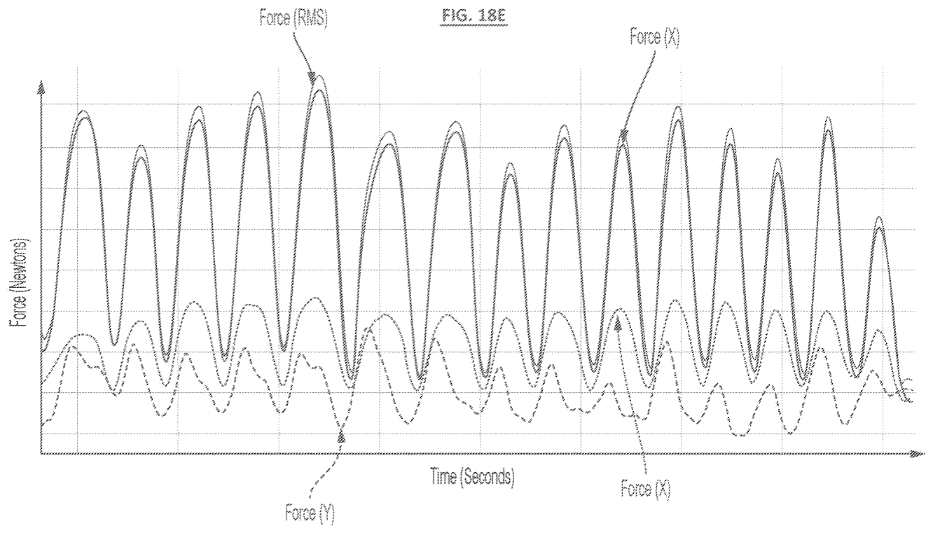

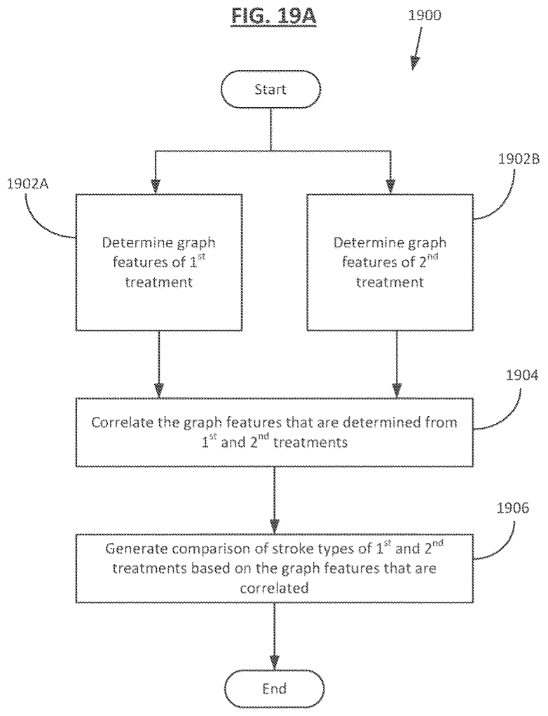

FIGS. 17A through 17E show different physical manipulation motion patterns which may be applied during treatment, and FIGS. 18A through 18E show their respective 3D force vs time waveform patterns as measured and generated using the system as disclosed herein.

FIGS. 17A and 18A pertain to a linear motion of the device 4, which may be referred to as a cross-fiber massage, in which forward and backward motions (longitudinal) are applied using a dispersive force-motion applicator (e.g., with a broader convex steel treatment edge for tissue palpation or scanning and treatment) using double-handed sustained pressure. Compressive force components peaks “Force (Z)” are almost aligned in phase with the peaks of the longitudinal motions “Force (Y)” and resultant force “Force (RMS)”. For most linear motion forces, there is a synchronous positive or negative swing of the Y axis component with respect to Z axis component depending on the forward or backward tilt, while the latitudinal component “Force (X)” is mostly at the zero crossing, unless the device 4 is tilted sideways.

FIGS. 17B and 18B pertain to a curvilinear motion of the device 4, also referred to as a fanning pattern, in which a curvilinear fanning motion is performed by pivoting one edge of the device 4, which is a dispersive applicator. The peaks of force components “Force (Z)” and “Force (Y)” are aligned out of phase, asynchronous to each other. The deflection of force along the Y direction, i.e. the peaks of “Force (Y)”, are aligned closer to a stroke valley, i.e. the valleys of “Force (Z)”, as evident from the adjacent figure of waveform. The deflection may turn the other way if the direction of the motion is changed from clockwise to anti clockwise. As shown, one edge is pivoted to fan the device 4 across a wide area.

FIGS. 17C and 18C pertain to a skin rolling motion, where the contour of the rolled skin is shown in waves in the photograph as the user uses the device 4, which is a dispersive applicator, to apply a forward motion with a single-handed grip while rolling the tissue or skin with the other hand to produce a longitudinal tensile stress. The device 4 traverses through the waves of the skin with longer stroke length, sustained compression, and tensile force in the forward direction shown by the arrow. Hence, this stroke motion is of very low frequency (e.g., from 0.04 Hz to 0.5 Hz). The waveform shown depicts a single stroke motion pattern which continued for about 25 seconds. The deflections in the “Force (Y)” component are due to the restrictions of the bumpy ride of the device in the forward direction. Both “Force (Z)” and “Force (X)” force components detect irregularities, as shown.

FIGS. 17D and 18D pertain to a linear motion of the device 4 which is a localized applicator (e.g., with a half-disc shaped tapered tooltip) along a measured stroke length. The forward and backward motion (longitudinal) is applied as shown by the arrows by holding the device 4 using pencil grip and using a stroke length of about 1 inch. The waveform shows a high frequency (approximately 3 Hz) linear motion. The uniformity of the waveform as shown by the peak-peak correspondence of the compressive component “Force (Z)” and the latitudinal component “Force (X)” show linearity in motion. Although the longitudinal component “Force (Y)” is out of phase by 180 degrees, its alignment of valleys with peaks of “Force (X)” and “Force (Z)” components shows uniformity and depicts linear motion.

FIGS. 17E and 18E pertain to a curvilinear motion of the device 4 which is a localized applicator, also referred to as a J stroke due to the motion resembling the letter J. The curvilinear motion implements a palm-based handgrip by the user, as shown. The latitudinal force component “Force (X)”, as tilted towards right, resembles positive deflection in the waveforms. The positive “Force (X)” force component and the compressive force component “Force (Z)” are in phase, but the longitudinal “Force (Y)” force component is out of phase with positive deflections of peaks closer to the valleys of the resultant force “Force (RMS)”. This shows a turning motion in the waveform.

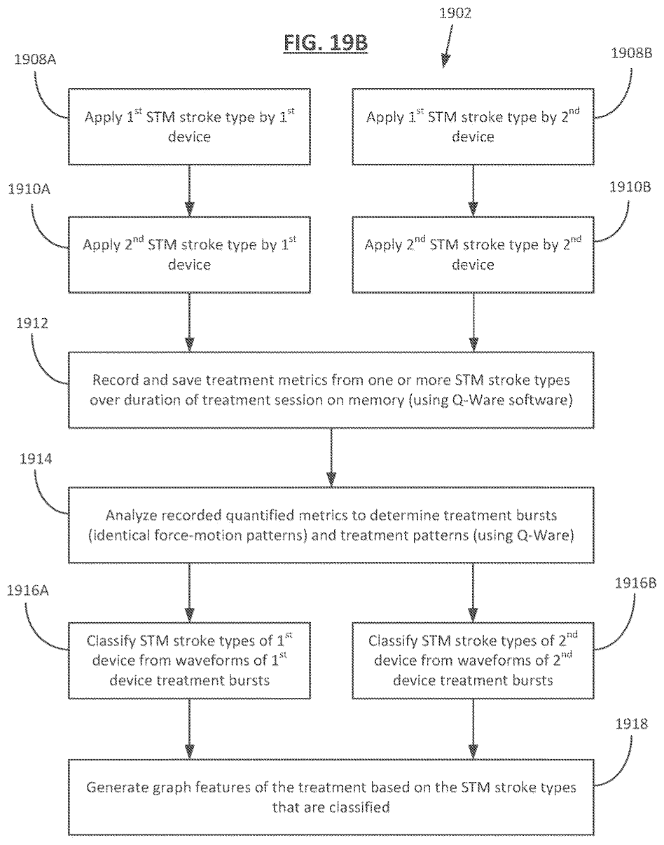

FIGS. 19A and 19B show a process 1900 implemented by the system according to embodiments disclosed herein. In step 1902A of FIG. 19A, the graph features of the first treatment are determined using the process 1902 as shown in FIG. 19B. In step 1902B of FIG. 19A, the graph features of the second treatment are determined using the process 1902 as shown in FIG. 19B. In FIG. 19A, the second treatment may be performed by the same practitioner as the one associated with the first treatment for an intra-therapist treatment report comparison, or by a different practitioner from the first treatment for an inter-therapist treatment report comparison. It should be understood that, although there are only first and second treatments disclosed in the process, there may be additional treatment(s) which may be analyzed and classified, i.e. a third treatment, a fourth treatment, a fifth treatment, etc., as suitable.

Referring to FIG. 19B, the process 1902 includes steps 1908A and 1908B, in which the first STM stroke type is applied by two devices (first and second). For example, in steps 1910A and 1910B, the second STM stroke type is applied by the two devices (first and second); that is, the first device is used to apply the first STM stroke type in step 1908A, and the second device is used to apply the same first STM stroke type in step 1908B. In some implementations, these first and second devices may be the same device that is used to apply the STM stroke type, as suitable.

In step 1912, the treatment metrics data from the one or more STM stroke types are recorded in the memory unit over a duration of treatment session using software such as the Q-Ware software. In step 1914, the recorded quantified metrics are analyzed to determine treatment bursts (such as the identical force-motion patterns) and treatment patterns, using the software.

In steps 1916A and 1916B, the STM stroke types are classified. For example, the STM stroke types of the first device is classified based on the waveforms, e.g. the multimodal graphical waveforms, of the first device treatment bursts in step 1916A, and the STM stroke types of the second device is classified from the waveforms of the second device treatment bursts in step 1916B. Subsequently, in step 1918, these STM stroke types that are classified are used to generate graph features of the treatment as performed using the respective devices. In some examples, the generation of the graph features also includes, prior to generating the graph features in step 1918, a determination or identification of whether the first and second STM stroke patterns are identical, based on the STM stroke types that are classified as a result of performing steps 1916A and 1916B, such that the identical STM stroke patterns can be correlated in step 1904 of FIG. 19A as further explained herein.

The steps of process 1902 in FIG. 19B are repeated for each of the first treatment (step 1902A of FIG. 19A) and the second treatment (step 1902B of FIG. 19A) such that the graph features of both the first and second treatments are generated based on the classified STM stroke types.

In step 1904 of FIG. 19A which follows step 1918 of FIG. 19B, the graph features that are determined from the first and second treatments in steps 1902A and 1902B (that is, as result of classifying temporal force-motion waveforms into identical soft tissue manipulation patterns or STM stroke types) are correlated using any suitable means as disclosed herein, such as peak-to-peak correlation, phase correlation, etc. Thereafter, in step 1906, the comparison of the STM stroke types of the first and second treatments is generated based on the graph features that are correlated in step 1904. The generated comparison may be displayed on a visual display for analysis by the practitioner(s), as suitable. For example, the visual comparison of these waveforms may be displayed such that the waveforms are superimposed on each other to identify a degree of variability or similarity between the waveforms representing individual or identical soft-tissue manipulation stroke types performed by a same practitioner or different practitioners. In some examples, individual soft-tissue manipulation stroke types may be classified in the aforementioned step(s) as force-motion signature patterns of the waveforms, based on the graphical features generated in the waveforms associated with the soft-tissue manipulation stroke type(s), and the classified force-motion signature patterns of two separate stroke types may be identified as being identical with or different from each other. Thereafter, a percentage of match of the waveforms of these stroke types may be generated in response to identifying that the stroke types are identical, based on the degree of variability or similarity estimated in the graphical features of the waveforms.

In some examples, the QSTM system 2 provides, via the interactive display using the GVI, a real-time guide for the practitioner during a soft-tissue manipulation treatment session to maintain a target force consistency by setting a target force trendline. The QSTM device(s) 4 may record quantifiable metrics associated with a plurality of soft-tissue manipulation stroke types applied. The quantifiable metrics are measured by the QSTM device 4 associated in a single-device treatment session or a multiple-device treatment session. In multiple-device treatment session, the remote device 1301 may display a visual feedback, automatically detect which one of the multiple QSTM devices is in use, and switch (based on the detecting without any user input) a device-specific user interface (e.g. via the GVI) to display a live (i.e., real-time) animated graphical visualization. The remote device 1301 may also generate a composite report of the soft-tissue manipulation treatment involving the QSTM devices 4 for the multiple-device treatment session, wherein the report captures a sequence of treatments in an order of the QSTM devices 4 that are used during the soft-tissue manipulation treatment. Using the QSTM devices 4 and the remote device 1301, the QSTM system 2 may classify the plurality of soft-tissue manipulation stroke types performed by the practitioner, and determine whether the soft-tissue manipulation stroke types as classified are identical to a plurality of stroke types determined from a history of treatment reports which includes force-motion waveform data representing historical soft-tissue manipulation stroke types that are previously recorded. The system 2 may generate a current force-motion waveform data representing the soft-tissue manipulation stroke types and a historical force-motion waveform data representing the historical soft-tissue manipulation stroke types that are considered identical to the soft-tissue manipulation stroke types. The system 2 may also analyze the current and historical force-motion waveform data to determine a degree of variability between the current and historical force-motion waveform data as disclosed herein, such that the degree of variability is represented as a percentage match.

The handheld force-motion tracking medical device along with a corresponding software program, for example the user-friendly operating software Q-Ware, as described in examples of this disclosure, characterize clinical manual therapy treatments in the form of QSTM. For example, the corresponding visual graphics on Q-Ware may identify a variety of visually distinguishable force-motion patterns applied in manual therapy treatment, for pain assessment and treatment replication. Both the device firmware and Q-Ware on PC may be robust and reliable, as the variable frame rate of GVI in Q-Ware during real-time data-visualization optimizes response time and data storage. GVI in Q-Ware may offer the user to set a “Target Force Trendline”, during treatment, with which the user can apply targeted peak force per stroke cycle during application while visually monitoring the PC screen. The 3D force-motion waveforms, as recorded during treatment sessions of LBP, may unveil identical signatures of linear or curvilinear stroke patterns which are applied in different directions by the clinician. According to some examples, a clinical assessment of the case study performed on the human subject with low back pain showed promising results with gradual progression in flexibility, soft tissue quality, and pressure pain tolerance of the subject leading to self-reported pain reduction. Thus, QSTM technology or system, according to some examples, not only offers objective metrics to quantify manual therapy but also presents means to advance state-of-the-art practice and a common language for manual therapy prescription. Such technology or system may be beneficial in facilitating device precision especially in the areas of (a) adaptive self-calibration; (b) pose estimation and orientation tracking; (c) treatment burst identification; and (d) estimating the device location during dynamic force-motion applications, for example.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

The foregoing discussion has been presented for purposes of illustration and description. The foregoing is not intended to limit the disclosure to the form or forms disclosed herein. In the foregoing Summary for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the description has included description of one or more aspects, embodiments, and/or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A Quantifiable Soft Tissue Manipulation (QSTM) system comprising:
at least one QSTM device for quantifying soft tissue manipulation, wherein the QSTM device is configured to apply a massage therapy on a soft tissue of a patient, wherein the massage therapy includes one or more soft-tissue manipulation stroke types applied by a single practitioner during a treatment session, wherein the QSTM device is a handheld mechatronic force-motion applicator,
the QSTM device comprising:
at least one treatment edge,
at least one force or motion sensing unit mechanically coupled to the at least one treatment edge and configured to measure quantifiable metrics including 3D motions and magnitude of compressive and shear forces applied during directional hand movements of the QSTM device for the soft-tissue manipulation by performing the one or more soft-tissue manipulation stroke types using the at least one treatment edge,
a processing unit coupled with the at least one force or motion sensing unit to compute resultant (RMS) force data from the measured compressive and shear forces, and angular orientation data from the 3D motions including one or more of: linear acceleration, angular velocity, or compass direction,
a memory unit configured to store sensor calibration information and to record data of the compressive and shear forces as measured by the at least one force or motion sensing unit, and treatment timestamps, in order to quantify the one or more soft-tissue manipulation stroke types performed on the patient,
a control button to be operated by the practitioner to change operation modes or working states of the QSTM device during the treatment session,
RGB-LED lights to visualize the operation modes and the working states of the QSTM device, and
a transmitter-receiver for serial communication and transmission of the RMS force and angular orientation data, the treatment timestamps, and the data of the compressive and shear forces via data stream to a remote device.

2. The system of claim 1, further comprising the remote device with an interactive visual display having multiprocessing computational and memory capacities, the remote device configured to:
receive the data of the compressive and shear forces, the RMS force and angular orientation data, and the treatment timestamps transmitted from the at least one QSTM device;
generate, based on the RMS force and angular orientation data, graphical data in form of multimodal graphical waveforms for a visual, numeric, or statistical comparison of the quantifiable metrics associated with the one or more soft-tissue manipulation stroke types performed by the practitioner during the treatment session; and
execute a software program for QSTM-based electronic treatment record to generate treatment reports and to document the treatment sessions.

3. The system of claim 2, wherein the remote device is configured to save and record the generated treatment reports and the quantifiable metrics of the massage therapy in one or more treatment sessions performed by one or more practitioner on corresponding patients for treatment data organization and maintenance.

4. The system of claim 2 wherein the remote device is further configured to determine one or more burst counts of a progression of soft-tissue manipulation strokes from the multimodal graphical waveforms and corresponding stroke counts applied in different stroke frequencies as a part of the treatment reports of the treatment session.

5. The system of claim 4, wherein each of the burst counts is determined based on one or more of: (a) computed 3D force measurements including compressive, shear lateral, and shear longitudinal force measurements, (b) angular motion measurements including yaw, pitch, and roll measurements, (c) data of the 3D motions including 3D accelerometer values, 3D gyroscope values, or 3D magnetometer values, or (d) start and stop timestamps of the treatment session.

6. The system of claim 4, wherein each of the stroke counts is determined based on thresholds of a decision tree comprising one or more of: computed 3D force measurements including compressive, shear lateral, and shear longitudinal force measurements, or start and stop timestamps associated with the burst counts.

7. The system of claim 2, wherein the generated graphical data includes a first multimodal graphical waveform representing a first soft-tissue manipulation stroke type and a second multimodal graphical waveform representing a second soft-tissue manipulation stroke type, and the visual comparison is displayed such that the first multimodal graphical waveform is superimposed on the second multimodal graphical waveform to identify a degree of variability or similarity between the multimodal graphical waveforms representing individual or identical soft-tissue manipulation stroke types performed by a same practitioner or different practitioners.

8. The system of claim 7, wherein the remote device is further configured to:
classify individual soft-tissue manipulation stroke types as force-motion signature patterns of the multimodal graphical waveforms, based on the graphical features generated in the multimodal graphical waveforms associated with the plurality of soft-tissue manipulation stroke types;
identify that the classified force-motion signature patterns of a first tissue manipulation stroke type and a second soft-tissue manipulation stroke type are identical or different; and
generate a percentage of match of the multimodal graphical waveforms of the first and second soft-tissue manipulation stroke types in response to identifying that the first and second soft-tissue manipulation stroke types are identical based on the degree of variability or similarity estimated in the graphical features of the multimodal graphical waveforms.

9. The system of claim 1, wherein the system further comprises:
a first QSTM device for recording and quantifying a first treatment sub-session by facilitating the practitioner to apply the one or more soft-tissue manipulation stroke types using the first QSTM device and
a second QSTM device for recording and quantifying a second treatment sub-session by facilitating the practitioner to apply the one or more soft-tissue manipulation stroke types using the second QSTM device,
wherein the first and second QSTM devices are used one after another in sequential repetition by the same practitioner to perform a multiple-device treatment session covering regional areas of the body,
wherein the practitioner is prompted to document treatment remarks about the treatment sub-sessions on the remote device via an interactive visual display before saving the treatment report of the performed treatment session.

10. The system of claim 9, wherein the remote device is configured to display a visual feedback, automatically detect which one of the first and second QSTM devices is in use, and switch, based on the detecting without any user input, a device-specific user interface to display a live animated graphical visualization.

11. The system of claim 1, wherein at least one QSTM device facilitates the practitioner to apply the one or more soft-tissue manipulation stroke types by the same QSTM device performing a single-device treatment session.

12. The system of claim 1, wherein the system further comprises a first QSTM device and a second QSTM device,
wherein the system facilitates a multiple-device treatment session by the practitioner in which a first soft-tissue manipulation is performed using the first QSTM device and a second soft-tissue manipulation is performed using the second QSTM device by the same practitioner after completion of the first soft-tissue manipulation, and
each of the first and second QSTM devices includes a microprocessor and a memory unit operatively coupled therewith, the memory unit storing instructions thereon which, when run on the microprocessor, cause the microprocessor to:
detect that the first soft-tissue manipulation is completed and the first QSTM device is in a rest state or placed on its cradle; and
perform, based on the detecting that the first soft-tissue manipulation is completed, an automatic self-calibration on the at least one sensor of the first QSTM device before its second use in the same treatment session.

13. A method of guiding and analyzing a soft-tissue manipulation treatment performed by one or more practitioners, the method comprising:
providing, by the remote device with an interactive display, a real-time guide for the practitioner during a soft-tissue manipulation treatment session to maintain a target force consistency by setting a target force trendline;
recording, by a processing unit, quantifiable metrics associated with a plurality of soft-tissue manipulation stroke types applied using handheld QSTM devices for quantifying the soft-tissue manipulation treatment, the quantifiable metrics being measured by at least one QSTM device associated in a single-device treatment session or a multiple-device treatment session; and
generating, based on the quantifiable metrics that are recorded, a composite report of the soft-tissue manipulation treatment involving the QSTM devices for the multiple-device treatment session, wherein the report captures a sequence of treatments in an order of the QSTM devices that are used during the soft-tissue manipulation treatment.

14. The method of claim 13, further comprising:
classifying the plurality of soft-tissue manipulation stroke types performed by the practitioner using the QSTM devices;
determining whether the soft-tissue manipulation stroke types as classified are identical to a plurality of stroke types determined from a history of treatment reports, wherein the history of treatment reports includes force-motion waveform data representing historical soft-tissue manipulation stroke types that are previously recorded;
generating a current force-motion waveform data representing the soft-tissue manipulation stroke types and a historical force-motion waveform data representing the historical soft-tissue manipulation stroke types that are considered identical to the soft-tissue manipulation stroke types; and
analyzing the current and historical force-motion waveform data to determine a degree of variability between the current and historical force-motion waveform data, wherein the degree of variability is represented as a percentage match.
